(19) United States
(12) Patent Application Publication
(10) Pub. No.: US 2008/0139581 A1
(43) Pub. Date: Jun. 12, 2008

(54) 2-Substituted 7-Aminoazolopyrimidines, Processes For Their Preparation And Their Use For Controlling Harmful Fungi, And Compositions Comprising These Compounds

(75) Inventors: Wassilios Grammenos, Ludwigshafen (DE); Thomas Grote, Wachenheim (DE); Udo Hunger, Mannheim (DE); Jan Klaas Lohmann, Mannheim (DE); Bern Muller, Frankenthal (DE); Joachim Rheinheimer, Ludwigshafen (DE); Peter Schafer, Ottersheim (DE); Anja Schwogler, Mannheim (DE); Michael Rack, Eppelheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/885,536

(22) PCT Filed: Mar. 2, 2006
(86) PCT No.: PCT/EP06/60399

§ 371 Date: Aug. 30, 2007

(30) Foreign Application Priority Data
Mar. 2, 2005 (DE) 102005010110.0
May 17, 2005 (DE) 102005023388.0

(51) Int. Cl. A01N 43/90; A01P 3/00; C07D 487/04

(52) U.S. Cl. 514/259.31; 544/263; 544/281; 514/259.3

(57) ABSTRACT

2-Substituted 7-aminoazolopyrimidines of the formula I

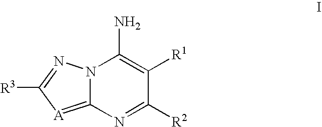

in which the substituents are as defined below:
R¹ is hydrogen, halogen, cyano, alkyl, haloalkyl, alkenyl, alkynyl, cycloalkyl, alkoxy, alkoxyalkyl, benzyloxyalkyl, alkoxyalkenyl or alkoxyalkynyl;
R² is hydrogen, halogen, cyano, alkyl, haloalkyl, alkenyl, alkynyl, cycloalkyl, alkoxy, alkoxyalkyl and alkylthioalkyl,
   where the carbon chains in R¹ and/or R² may be substituted according to the description;
R³ is halogen, cyano, NR^{A}R^{B}, hydroxyl, mercapto, alkyl, haloalkyl, cycloalkyl, alkoxy, alkylthio, cycloalkoxy, cycloalkylthio, carboxyl, formyl, alkylcarbonyl, alkoxycarbonyl, alkenyloxycarbonyl, alkynyloxycarbonyl, phenyl, phenoxy, phenylthio, benzyloxy, benzylthio, alkyl-S(O)ₘ—;
A is N and CR^{x};
   R^{x} is hydrogen or one of the groups mentioned under R³;
      processes and intermediates for preparing these compounds, compositions comprising them and their use for controlling phytopathogenic harmful fungi.

The present invention relates to 2-substituted 7-aminoazolopyrimidines of the formula I

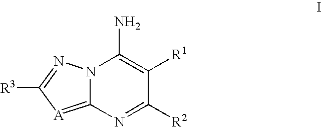

in which the substituents are as defined below:
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl and C₁-C₁₂-alkylthio-C₁-C₁₂-alkyl,
   where the carbon chains in R¹ and/or R² may be substituted by one to four identical or different groups R^{a}:
   R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, NR^{A}R^{B}, phenyl, C₁-C₆-alkylphenyl;
      R^{A}, R^{B} are hydrogen and C₁-C₆-alkyl;
      where the cyclic groups in R^{a} may be substituted by one to four groups R^{b}:
      R^{b} is halogen, cyano, hydroxyl, mercapto, C₁-C₁₀-alkyl, C₁-C₁₀-haloalkyl, C₂-C₁₀-alkenyl, C₂-C₁₀-alkynyl and C₁-C₆-alkoxy;
R³ is halogen, cyano, NR^{A}R^{B}, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—;
   m is 0, 1 or 2; and
A is N and CR^{x};
   R^{x} is hydrogen or one of the groups mentioned under R³.

Moreover, the invention relates to processes for preparing these compounds, to compositions comprising them and to their use for controlling phytopathogenic harmful fungi.

5,6-Dialkyl-7-aminoazolopyrimidines are proposed in a general manner in GB 1 148 629. Individual fungicidally active 5,6-dialkyl-7-aminoazolopyrimidines are known from EP-A 141 317. However, in many cases their activity is unsatisfactory. Based on this, it is an object of the present invention to provide compounds having improved activity and/or a wider activity spectrum.

We have found that this object is achieved by the compounds defined at the outset. Furthermore, we have found processes and intermediates for their preparation, compositions comprising them and methods for controlling harmful fungi using the compounds I.

The compounds of the formula I differ from those in the abovementioned publications by the specific embodiment of the substituent in the 2-position of the azolopyrimidine skeleton.

Compared to the known compounds, the compounds of the formula I are more effective against harmful fungi.

The compounds according to the invention can be obtained by different routes. Advantageously, the compounds according to the invention are obtained by converting substituted β-ketoesters of the formula II with an aminoazole of the formula III to give 7-hydroxyazolopyrimidines of the formula IV. The variables in formulae II and IV are as defined for formula I and the group R in formula II is C₁-C₄-alkyl; for practical reasons, preference is given here to methyl, ethyl or propyl.

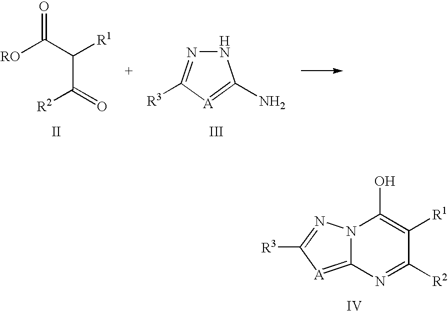

The compounds of the formula IV are novel.

The reaction of the substituted β-ketoesters of the formula II with the aminoazoles of the formula III can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines and mixtures of these solvents with water. Suitable catalysts are bases as mentioned above or acids such as sulfonic acids or mineral acids. With particular preference, the reaction is carried out in the absence of a solvent or in chlorobenzene, xylene, dimethyl sulfoxide or N-methylpyrrolidone. Particularly preferred bases are tertiary amines, such as triisopropylethylamine, tributylamine, N-methylmorpholine or N-methylpiperidine. The temperatures are from 50 to 300° C., preferably from 50 to 180° C., if the reaction is carried out in solution [cf. EP-A 770 615; Adv. Het. Chem. 57 (1993), 81ff].

The bases are generally employed in catalytic amounts; however, they can also be employed in equimolar amounts, in excess or, if appropriate, as solvent.

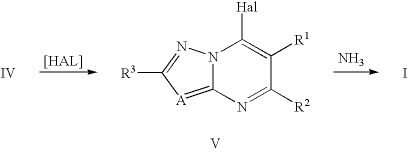

In most cases, the resulting condensates of the formula IV precipitate from the reaction solutions in pure form and, after washing with the same solvent or with water and subsequent drying they are reacted with halogenating agents, in particular chlorinating or brominating agents, to give the compounds of the formula V in which Hal is chlorine or bromine, in particular chlorine. The reaction is preferably carried out using chlorinating agents such as phosphorus oxychloride, thionyl chloride or sulfuryl chloride at from 50° C. to 150° C., preferably in excess phosphorus oxytrichloride at reflux temperature. After evaporation of excess phosphorus oxytrichloride, the residue is treated with ice-water, if appropriate with addition of a water-immiscible solvent. In most cases, the chlorinated product isolated from the dried organic phase, if appropriate after evaporation of the inert solvent, is very pure and is subsequently reacted with ammonia in inert solvents at from 100° C. to 200° C. to give the 7-aminoazolo[1,5-a]pyrimidines. The reaction is preferably carried out using a 1- to 10-molar excess of ammonia, under a pressure of from 1 to 100 bar.

The novel 7-aminoazolo[1,5-a]pyrimidines are, if appropriate after evaporation of the solvent, isolated as crystalline compounds, by digestion in water.

The β-ketoesters of the formula II can be prepared as described in Organic Synthesis Coll. Vol. 1, p. 248, and/or they are commercially available.

The intermediates of the formula V are novel.

Alternatively, the novel compounds of the formula I can be obtained by reacting substituted acyl cyanides of the formula VI in which R¹ and R² are as defined above with an aminoazole of the formula III.

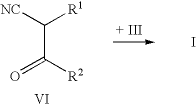

The reaction can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as those mentioned above, and mixtures of these solvents with water. The reaction temperatures are from 50 to 300° C., preferably from 50 to 150° C., if the reaction is carried out in solution.

The novel 7-aminoazolo[1,5-a]pyrimidines are, if appropriate after evaporation of the solvent or dilution with water, isolated as crystalline compounds.

Some of the substituted alkyl cyanides of the formula VI required for preparing the 7-aminoazolo[1,5-a]pyrimidines are known, or they can be prepared by known methods from alkyl cyanides and carboxylic acid esters using strong bases, for example alkali metal hydrides, alkali metal alkoxides, alkali metal amides or metal alkyls (cf.: J. Amer. Chem. Soc. 73 (1951), p. 3766).

Compounds of the formula I in which R^{x} is C₁-C₁₄-haloalkyl, C₁-C₁₂-haloalkoxy-C₁-C₁₂-alkyl, C₁-C₁₂-alkoxy-C₁-C₁₂-haloalkyl, C₂-C₁₂-haloalkenyl or C₂-C₁₂-haloalkynyl can be obtained by halogenation of corresponding halogen-free azolopyrimidines of the formula I, they are referred to as compounds I′. In formula I′, R^{1′} is a halogen-free group R¹. In formula I″, R^{1″} is a halogenated group R¹:

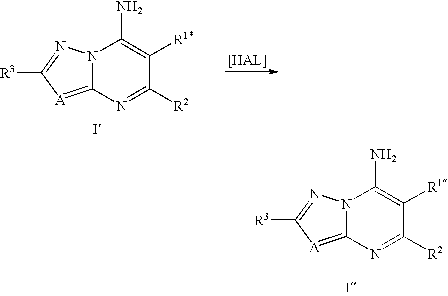

The halogenation is usually carried out at temperatures of from 0° C. to 200° C., preferably from 20° C. to 110° C., in an inert organic solvent in the presence of a free-radical initiator (for example dibenzoyl peroxide or azobisisobutyronitrile or under UV irradiation using, for example, an Hg vapor lamp) or an acid [cf. Synthetic Reagents, Vol. 2, pp. 1-63, Wiley Publishers, New York (1974)].

The reaction partners are generally reacted with one another in equimolar amounts. In terms of yield, it may be advantageous to employ an excess of halogenating agent, based on 1′.

Suitable halogenating agents are, for example, elemental halogens (for example Cl₂, Br₂, I₂), N-bromosuccinimide, N-chlorosuccinimide oder dibromodimethylhydrantoin. The halogenating agents are generally employed in equimolar amounts, in excess or, if appropriate, as solvent.

If individual compounds I cannot be obtained by the routes described above, they can be prepared by derivatization of other compounds I.

If the synthesis yields mixtures of isomers, a separation is generally not necessarily required, however, since in some cases the individual isomers can be interconverted during work-up for use or during application (for example under the action of light, acids or bases). Such conversions may also take place after use, for example in the treatment of plants in the treated plants, or in the harmful fungus to be controlled.

In the definitions of symbols given in the formulae above, collective terms were used which are generally representative of the following substituents:

halogen: fluorine, chlorine, bromine and iodine, in particular fluorine or chlorine;
alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6, 8 or 10 carbon atoms and one or two double bonds in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkoxyalkyl: a saturated straight-chain or mono-, di- or tribranched hydrocarbon chain which is interrupted by an oxygen atom, for example C₅-C₁₂-alkoxyalkyl: a hydrocarbon chain as described above having 5 to 12 carbon atoms which may be interrupted by an oxygen atom in any position, such as propoxyethyl, butoxyethyl, pentoxyethyl, hexyloxyethyl, heptyloxyethyl, octyloxyethyl, nonyloxyethyl, 3-(3-ethylhexyloxy)ethyl, 3-(2,4,4-trimethylpentyloxy)ethyl, 3-(1-ethyl-3-methylbutoxy)ethyl, ethoxypropyl, propoxypropyl, butoxypropyl, pentoxypropyl, hexyloxypropyl, heptyloxypropyl, octyloxypropyl, nonyloxypropyl, 3-(3-ethylhexyloxy)propyl, 3-(2,4,4-trimethylpentyloxy)propyl, 3-(1-ethyl-3-methylbutoxy)propyl, ethoxybutyl, propoxybutyl, butoxybutyl, pentoxybutyl, hexyloxybutyl, heptyloxybutyl, octyloxybutyl, nonyloxybutyl, 3-(3-ethylhexyloxy)butyl, 3-(2,4,4-trimethylpentyloxy)butyl, 3-(1-ethyl-3-methylbutoxy)butyl, methoxypentyl, ethoxypentyl, propoxypentyl, butoxypentyl, pentoxypentyl, hexyloxypentyl, heptyloxypentyl, 3-(3-methylhexyloxy)pentyl, 3-(2,4-dimethylpentyloxy)pentyl, 3-(1-ethyl-3-methylbutoxy)pentyl;
haloalkenyl: unsaturated straight-chain or branched hydrocarbon radicals having 2 to 10 carbon atoms and one or two double bonds in any position (as mentioned above), where some or all of the hydrogen atoms in these groups may be replaced by halogen atoms as mentioned above, in particular by fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 carbon ring members, such as cyclopropyl, cyclobutyl, cyclopentyl and cyclohexyl.

The scope of the present invention includes the (R)- and (S)-isomers and the racemates of compounds of the formula I having chiral centers.

With a view to the intended use of the azolopyrimidines of the formula I, particular preference is given to the following meanings of the substituents, in each case on their own or in combination:

Preference is given to compounds I in which the group R¹ has at most 9 carbon atoms. Preference is likewise given to compounds of the formula I in which the groups R¹ and R² together have at most 14 carbon atoms.

In one embodiment of the compounds I according to the invention, R¹ and R² independently of one another are halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₆-alkoxy-C₁-C₆-alkyl, where the carbon chains in R¹ and/or R² may be substituted by one to four identical or different of the following groups R^{a}:
halogen, cyano, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy-C₁-C₆-alkyl, phenyl which may be substituted by an alkyl group.

In a preferred embodiment of the compounds I according to the invention, R¹ and R² independently of one another are C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₆-alkoxy-C₁-C₆-alkyl, where the carbon chains in R¹ and/or R² may be substituted as described above.

Particular preference is given to those compounds I in which R² is C₁-C₅-alkyl, C₁-C₅-haloalkyl, C₂-C₅-alkenyl, C₂-C₅-alkynyl, C₃-C₅-cycloalkyl, C₁-C₅-alkoxy, C₁-C₄-alkoxy-C₁-C₄-alkyl, which groups are unsubstituted or substituted by halogen, cyano, methyl or ethyl.

In a further preferred embodiment of the compounds I according to the invention, R² is C₁-C₅-alkyl, C₁-C₅-haloalkyl, C₂-C₅-alkenyl, C₂-C₅-alkynyl, C₃-C₅-cycloalkyl, C₁-C₅-alkoxy, C₁-C₄-alkoxy-C₁-C₄-alkyl, which groups are unsubstituted or substituted by halogen, cyano, methyl or ethyl.

In a further particularly preferred embodiment of the compounds I according to the invention,
R¹ is C₁-C₁₂-alkyl, C₁-C₁₂-haloalkyl, C₂-C₁₂-alkenyl, C₃-C₁₂-alkynyl, C₁-C₆-alkoxy-C₁-C₆-alkyl, and
R² is C₁-C₅-alkyl, C₁-C₅-haloalkyl, C₂-C₅-alkenyl, C₂-C₅-alkynyl, C₁-C₄-alkoxy-C₁-C₄-alkyl,
   where the carbon chains in R¹ and/or R² may be partially or fully halogenated or substituted by C₂-C₅-alkenyl or C₂-C₅-alkynyl.

In a further particularly preferred embodiment of the compounds I according to the invention, R² is C₁-C₅-alkyl, C₁-C₅-haloalkyl, C₂-C₅-alkenyl, C₂-C₅-alkynyl, C₁-C₄-alkoxy-C₁-C₄-alkyl, where the carbon chains in R¹ and/or R² may be partially or fully halogenated.

In a preferred embodiment of the compounds of the formula I, group R^{a} is absent.

In one embodiment of the compounds I according to the invention, R² is methyl, ethyl, isopropyl, n-propyl or n-butyl, in particular methyl.

Halogen atoms in the groups R¹ and/or R² are preferably located at the α carbon atom.

Cyano groups in R¹ and/or R² are preferably located at the terminal carbon atom.

In a further preferred embodiment of the compounds of the formula I, group R^{b} is absent.

In a further preferred embodiment of the compounds I according to the invention, R³ is halogen, cyano, hydroxyl, mercapto, amino, C₂-C₆-alkyl, C₁-C₆-haloalkyl, C₃-C₈-cycloalkyl, C₁-C₆-alkoxy or C₁-C₆-alkylthio, in particular halogen or amines.

One embodiment of the compounds according to the invention relates to compounds I in which A is CR^{x}, in particular CH. These compounds correspond to formula I.1:

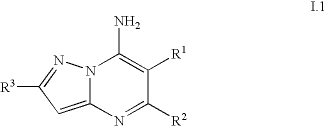

In addition, preference is also given to compounds I in which R^{x} is halogen, cyano, hydroxyl, mercapto, amino, C₂-C₆-alkyl, C₁-C₆-haloalkyl, C₃-C₈-cycloalkyl, C₁-C₆-alkoxy or C₁-C₆-alkylthio, in particular halogen, cyano and C₁-C₁₀-alkyl.

Another embodiment of the compounds according to the invention relates to compounds I in which A is N. These compounds correspond to formula I.2:

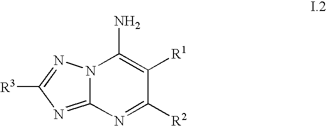

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

### Table 1

Compounds of the formula I.1 in which R¹ for each compound corresponds to one row of Table A, R² is methyl and R³ is fluorine

### Table 2

Compounds of the formula I.1 in which R¹ for each compound corresponds to one row of Table A, R² is ethyl and R³ is fluorine

### Table 3

Compounds of the formula I.1 in which R¹ for each compound corresponds to one row of Table A, R² is n-propyl and R³ is fluorine

### Table 4

Compounds of the formula I.1 in which R¹ for each compound corresponds to one row of Table A, R² is n-butyl and R³ is fluorine

### Table 5

Compounds of the formula I.1 in which R¹ for each compound corresponds to one row of Table A, R² is n-pentyl and R³ is fluorine

### Table 6

Compounds of the formula I.1 in which R¹ for each compound corresponds to one row of Table A, R² is n-hexyl and R³ is fluorine

### Table 7

Compounds of the formula I.1 in which R¹ for each compound corresponds to one row of Table A, R² is n-heptyl and R³ is fluorine

### Table 8

Compounds of the formula I.1 in which R¹ for each compound corresponds to one row of Table A, R² is n-octyl and R³ is fluorine

### Table 9

Compounds of the formula I.1 in which R¹ for each compound corresponds to one row of Table A, R² is n-nonyl and R³ is fluorine

### Table 10

Compounds of the formula I.1 in which R¹ for each compound corresponds to one row of Table A, R² is n-decyl and R³ is fluorine

### Table 11

Compounds of the formula I.1 in which R¹ for each compound corresponds to one row of Table A, R² is methoxymethyl and R³ is fluorine

### Table 12

Compounds of the formula I.1 in which R¹ for each compound corresponds to one row of Table A, R² is ethoxymethyl and R³ is fluorine

### Table 13

Compounds of the formula I.1 in which R¹ for each compound corresponds to one row of Table A, R² is methyl and R³ is chlorine

### Table 14

Compounds of the formula I.1 in which R¹ for each compound corresponds to one row of Table A, R² is ethyl and R³ is chlorine

### Table 15

Compounds of the formula I.1 in which R¹ for each compound corresponds to one row of Table A, R² is n-propyl and R³ is chlorine

### Table 16

Compounds of the formula I.1 in which R¹ for each compound corresponds to one row of Table A, R² is n-butyl and R³ is chlorine

### Table 17

Compounds of the formula I.1 in which R¹ for each compound corresponds to one row of Table A, R² is n-pentyl and R³ is chlorine

### Table 18

Compounds of the formula I.1 in which R¹ for each compound corresponds to one row of Table A, R² is n-hexyl and R³ is chlorine

### Table 19

Compounds of the formula I.1 in which R¹ for each compound corresponds to one row of Table A, R² is n-heptyl and R³ is chlorine

### Table 20

Compounds of the formula I.1 in which R¹ for each compound corresponds to one row of Table A, R² is n-octyl and R³ is chlorine

### Table 21

Compounds of the formula I.1 in which R¹ for each, compound corresponds to one row of Table A, R² is n-nonyl and R³ is chlorine

### Table 22

Compounds of the formula I.1 in which R¹ for each compound corresponds to one row of Table A, R² is n-decyl and R³ is chloride

### Table 23

Compounds of the formula I.1 in which R¹ for each compound corresponds to one row of Table A, R² is methoxymethyl and R³ is chlorine

### Table 24

Compounds of the formula I.1 in which R¹ for each compound corresponds to one row of Table A, R² is ethoxymethyl and R³ is chlorine

### Table 25

Compounds of the formula I.1 in which R¹ for each compound corresponds to one row of Table A, R² is methyl and R³ is bromine

### Table 26

Compounds of the formula I.1 in which R¹ for each compound corresponds to one row of Table A, R² is ethyl and R³ is bromine

### Table 27

Compounds of the formula I.1 in which R¹ for each compound corresponds to one row of Table A, R² is n-propyl and R³ is bromine

### Table 28

Compounds of the formula I.1 in which R¹ for each compound corresponds to one row of Table A, R² is n-butyl and R³ is bromine

### Table 29

Compounds of the formula I.1 in which R¹ for each compound corresponds to one row of Table A, R² is n-pentyl and R³ is bromine

### Table 30

Compounds of the formula I.1 in which R¹ for each compound corresponds to one row of Table A, R² is n-hexyl and R³ is bromine

### Table 31

Compounds of the formula I.1 in which R¹ for each compound corresponds to one row of Table A, R² is n-heptyl and R³ is bromine

### Table 32

Compounds of the formula I.1 in which R¹ for each compound corresponds to one row of Table A, R² is n-octyl and R³ is bromine

### Table 33

Compounds of the formula I.1 in which R¹ for each compound corresponds to one row of Table A, R² is n-nonyl and R³ is bromine

### Table 34

Compounds of the formula I.1 in which R¹ for each compound corresponds to one row of Table A, R² is n-decyl and R³ is bromine

### Table 35

Compounds of the formula I.1 in which R¹ for each compound corresponds to one row of Table A, R² is methoxymethyl and R³ is bromine

### Table 36

Compounds of the formula I.1 in which R¹ for each compound corresponds to one row of Table A, R² is ethoxymethyl and R³ is bromine

### Table 37

Compounds of the formula I.1 in which R¹ for each compound corresponds to one row of Table A, R² is methyl and R³ is cyano

### Table 38

Compounds of the formula I.1 in which R¹ for each compound corresponds to one row of Table A, R² is ethyl and R³ is cyano

### Table 39

Compounds of the formula I.1 in which R¹ for each compound corresponds to one row of Table A, R² is n-propyl and R³ is cyano

### Table 40

Compounds of the formula I.1 in which R¹ for each compound corresponds to one row of Table A, R² is n-butyl and R³ is cyano

### Table 41

Compounds of the formula I.1 in which R¹ for each compound corresponds to one row of Table A, R² is n-pentyl and R³ is cyano

### Table 42

Compounds of the formula I.1 in which R¹ for each compound corresponds to one row of Table A, R² is n-hexyl and R³ is cyano

### Table 43

Compounds of the formula I.1 in which R¹ for each compound corresponds to one row of Table A, R² is n-heptyl and R³ is cyano

### Table 44

Compounds of the formula I.1 in which R¹ for each compound corresponds to one row of Table A, R² is n-octyl and R³ is cyano

### Table 45

Compounds of the formula I.1 in which R¹ for each compound corresponds to one row of Table A, R² is n-nonyl and R³ is cyano

### Table 46

Compounds of the formula I.1 in which R¹ for each compound corresponds to one row of Table A, R² is n-decyl and R³ is cyano

### Table 47

Compounds of the formula I.1 in which R¹ for each compound corresponds to one row of Table A, R² is methoxymethyl and R³ is cyano

### Table 48

Compounds of the formula I.1 in which R¹ for each compound corresponds to one row of Table A, R² is ethoxymethyl and R³ is cyano

### Table 49

Compounds of the formula I.1 in which R¹ for each compound corresponds to one row of Table A, R² is methyl and R³ is hydroxyl

### Table 50

Compounds of the formula I.1 in which R¹ for each compound corresponds to one row of Table A, R² is ethyl and R³ is hydroxyl

### Table 51

Compounds of the formula I.1 in which R¹ for each compound corresponds to one row of Table A, R² is n-propyl and R³ is hydroxyl

### Table 52

Compounds of the formula I.1 in which R¹ for each compound corresponds to one row of Table A, R² is n-butyl and R³ is hydroxyl

### Table 53

Compounds of the formula I.1 in which R¹ for each compound corresponds to one row of Table A, R² is n-pentyl and R³ is hydroxyl

### Table 54

Compounds of the formula I.1 in which R¹ for each compound corresponds to one row of Table A, R² is n-hexyl and R³ is hydroxyl

### Table 55

Compounds of the formula I.1 in which R¹ for each compound corresponds to one row of Table A, R² is n-heptyl and R³ is hydroxyl

### Table 56

Compounds of the formula I.1 in which R¹ for each compound corresponds to one row of Table A, R² is n-octyl and R³ is hydroxyl

### Table 57

Compounds of the formula I.1 in which R¹ for each compound corresponds to one row of Table A, R² is n-nonyl and R³ is hydroxyl

### Table 58

Compounds of the formula I.1 in which R¹ for each compound corresponds to one row of Table A, R² is n-decyl and R³ is hydroxyl

### Table 59

Compounds of the formula I.1 in which R¹ for each compound corresponds to one row of Table A, R² is methoxymethyl and R³ is hydroxyl

### Table 60

Compounds of the formula I.1 in which R¹ for each compound corresponds to one row of Table A, R² is ethoxymethyl and R³ is hydroxyl

### Table 61

Compounds of the formula I.1 in which R¹ for each compound corresponds to one row of Table A, R² is methyl and R³ is mercapto

### Table 62

Compounds of the formula I.1 in which R¹ for each compound corresponds to one row of Table A, R² is ethyl and R³ is mercapto

### Table 63

Compounds of the formula I.1 in which R¹ for each compound corresponds to one row of Table A, R² is n-propyl and R³ is mercapto

### Table 64

Compounds of the formula I.1 in which R¹ for each compound corresponds to one row of Table A, R² is n-butyl and R³ is mercapto

### Table 65

Compounds of the formula I.1 in which R¹ for each compound corresponds to one row of Table A, R² is n-pentyl and R³ is mercapto

### Table 66

Compounds of the formula I.1 in which R¹ for each compound corresponds to one row of Table A, R² is n-hexyl and R³ is mercapto

### Table 67

Compounds of the formula I.1 in which R¹ for each compound corresponds to one row of Table A, R² is n-heptyl and R³ is mercapto

### Table 68

Compounds of the formula I.1 in which R¹ for each compound corresponds to one row of Table A, R² is n-octyl and R³ is mercapto

### Table 69

Compounds of the formula I.1 in which R¹ for each compound corresponds to one row of Table A, R² is n-nonyl and R³ is mercapto

### Table 70

Compounds of the formula I.1 in which R¹ for each compound corresponds to one row of Table A, R² is n-decyl and R³ is mercapto

### Table 71

Compounds of the formula I.1 in which R¹ for each compound corresponds to one row of Table A, R² is methoxymethyl and R³ is mercapto

### Table 72

Compounds of the formula I.1 in which R¹ for each compound corresponds to one row of Table A, R² is ethoxymethyl and R³ is mercapto

### Table 73

Compounds of the formula I.1 in which R¹ for each compound corresponds to one row of Table A, R² is methyl and R³ is amino

### Table 74

Compounds of the formula I.1 in which R¹ for each compound corresponds to one row of Table A, R² is ethyl and R³ is amino

### Table 75

Compounds of the formula I.1 in which R¹ for each compound corresponds to one row of Table A, R² is n-propyl and R³ is amino

### Table 76

Compounds of the formula I.1 in which R¹ for each compound corresponds to one row of Table A, R² is n-butyl and R³ is amino

### Table 77

Compounds of the formula I.1 in which R¹ for each compound corresponds to one row of Table A, R² is n-pentyl and R³ is amino

### Table 78

Compounds of the formula I.1 in which R¹ for each compound corresponds to one row of Table A, R² is n-hexyl and R³ is amino

### Table 79

Compounds of the formula I.1 in which R¹ for each compound corresponds to one row of Table A, R² is n-heptyl and R³ is amino

### Table 80

Compounds of the formula I.1 in which R¹ for each compound corresponds to one row of Table A, R² is n-octyl and R³ is amino

### Table 81

Compounds of the formula I.1 in which R¹ for each compound corresponds to one row of Table A, R² is n-nonyl and R³ is amino

### Table 82

Compounds of the formula I.1 in which R¹ for each compound corresponds to one row of Table A, R² is n-decyl and R³ is amino

### Table 83

Compounds of the formula I.1 in which R¹ for each compound corresponds to one row of Table A, R² is methoxymethyl and R³ is amino

### Table 84

Compounds of the formula I.1 in which R¹ for each compound corresponds to one row of Table A, R² is ethoxymethyl and R³ is amino

### Table 85

Compounds of the formula I.1 in which R¹ for each compound corresponds to one row of Table A, R² is methyl and R³ is trifluoromethyl

### Table 86

Compounds of the formula I.1 in which R¹ for each compound corresponds to one row of Table A, R² is ethyl and R³ is trifluoromethyl

### Table 87

Compounds of the formula I.1 in which R¹ for each compound corresponds to one row of Table A, R² is n-propyl and R³ is trifluoromethyl

### Table 88

Compounds of the formula I.1 in which R¹ for each compound corresponds to one row of Table A, R² is n-butyl and R³ is trifluoromethyl

### Table 89

Compounds of the formula I.1 in which R¹ for each compound corresponds to one row of Table A, R² is n-pentyl and R³ is trifluoromethyl

### Table 90

Compounds of the formula I.1 in which R¹ for each compound corresponds to one row of Table A, R² is n-hexyl and R³ is trifluoromethyl

### Table 91

Compounds of the formula I.1 in which R¹ for each compound corresponds to one row of Table A, R² is n-heptyl and R³ is trifluoromethyl

### Table 92

Compounds of the formula I.1 in which R¹ for each compound corresponds to one row of Table A, R² is n-octyl and R³ is trifluoromethyl

### Table 93

Compounds of the formula I.1 in which R¹ for each compound corresponds to one row of Table A, R² is n-nonyl and R³ is trifluoromethyl

### Table 94

Compounds of the formula I.1 in which R¹ for each compound corresponds to one row of Table A, R² is n-decyl and R³ is trifluoromethyl

### Table 95

Compounds of the formula I.1 in which R¹ for each compound corresponds to one row of Table A, R² is methoxymethyl and R³ is trifluoromethyl

### Table 96

Compounds of the formula I.1 in which R¹ for each compound corresponds to one row of Table A, R² is ethoxymethyl and R³ is trifluoromethyl

### Table 97

Compounds of the formula I.1 in which R¹ for each compound corresponds to one row of Table A, R² is methyl and R³ is ethyl

### Table 98

Compounds of the formula I.1 in which R¹ for each compound corresponds to one row of Table A, R² is ethyl and R³ is ethyl

### Table 99

Compounds of the formula I.1 in which R¹ for each compound corresponds to one row of Table A, R² is n-propyl and R³ is ethyl

### Table 100

Compounds of the formula I.1 in which R¹ for each compound corresponds to one row of Table A, R² is n-butyl and R³ is ethyl

### Table 101

Compounds of the formula I.1 in which R¹ for each compound corresponds to one row of Table A, R² is n-pentyl and R³ is ethyl

### Table 102

Compounds of the formula I.1 in which R¹ for each compound corresponds to one row of Table A, R² is n-hexyl and R³ is ethyl

### Table 103

Compounds of the formula I.1 in which R¹ for each compound corresponds to one row of Table A, R² is n-heptyl and R³ is ethyl

### Table 104

Compounds of the formula I.1 in which R¹ for each compound corresponds to one row of Table A, R² is n-octyl and R³ is ethyl

### Table 105

Compounds of the formula I.1 in which R¹ for each compound corresponds to one row of Table A, R² is n-nonyl and R³ is ethyl

### Table 106

Compounds of the formula I.1 in which R¹ for each compound corresponds to one row of Table A, R² is n-decyl and R³ is ethyl

### Table 107

Compounds of the formula I.1 in which R¹ for each compound corresponds to one row of Table A, R² is methoxymethyl and R³ is ethyl

### Table 108

Compounds of the formula I.1 in which R¹ for each compound corresponds to one row of Table A, R² is ethoxymethyl and R³ is ethyl

### Table 109

Compounds of the formula I.1 in which R¹ for each compound corresponds to one row of Table A, R² is methyl and R³ is n-propyl

### Table 110

Compounds of the formula I.1 in which R¹ for each compound corresponds to one row of Table A, R² is ethyl and R³ is n-propyl

### Table 111

Compounds of the formula I.1 in which R¹ for each compound corresponds to one row of Table A, R² is n-propyl and R³ is n-propyl

### Table 112

Compounds of the formula I.1 in which R¹ for each compound corresponds to one row of Table A, R² is n-butyl and R³ is n-propyl

### Table 113

Compounds of the formula I.1 in which R¹ for each compound corresponds to one row of Table A, R² is n-pentyl and R³ is n-propyl

### Table 114

Compounds of the formula I.1 in which R¹ for each compound corresponds to one row of Table A, R² is n-hexyl and R³ is n-propyl

### Table 115

Compounds of the formula I.1 in which R¹ for each compound corresponds to one row of Table A, R² is n-heptyl and R³ is n-propyl

### Table 116

Compounds of the formula I.1 in which R¹ for each compound corresponds to one row of Table A, R² is n-octyl and R³ is n-propyl

### Table 117

Compounds of the formula I.1 in which R¹ for each compound corresponds to one row of Table A, R² is n-nonyl and R³ is n-propyl

### Table 118

Compounds of the formula I.1 in which R¹ for each compound corresponds to one row of Table A, R² is n-decyl and R³ is n-propyl

### Table 119

Compounds of the formula I.1 in which R¹ for each compound corresponds to one row of Table A, R² is methoxymethyl and R³ is n-propyl

### Table 120

Compounds of the formula I.1 in which R¹ for each compound corresponds to one row of Table A, R² is ethoxymethyl and R³ is n-propyl

### Table 121

Compounds of the formula I.1 in which R¹ for each compound corresponds to one row of Table A, R² is methyl and R³ is methoxy

### Table 122

Compounds of the formula I.1 in which R¹ for each compound corresponds to one row of Table A, R² is ethyl and R³ is methoxy

### Table 123

Compounds of the formula I.1 in which R¹ for each compound corresponds to one row of Table A, R² is n-propyl and R³ is methoxy

### Table 124

Compounds of the formula I.1 in which R¹ for each compound corresponds to one row of Table A, R² is n-butyl and R³ is methoxy

### Table 125

Compounds of the formula I.1 in which R¹ for each compound corresponds to one row of Table A, R² is n-pentyl and R³ is methoxy

### Table 126

Compounds of the formula I.1 in which R¹ for each compound corresponds to one row of Table A, R² is n-hexyl and R³ is methoxy

### Table 127

Compounds of the formula I.1 in which R¹ for each compound corresponds to one row of Table A, R² is n-heptyl and R³ is methoxy

### Table 128

Compounds of the formula I.1 in which R¹ for each compound corresponds to one row of Table A, R² is n-octyl and R³ is methoxy

### Table 129

Compounds of the formula I.1 in which R¹ for each compound corresponds to one row of Table A, R² is n-nonyl and R³ is methoxy

### Table 130

Compounds of the formula I.1 in which R¹ for each compound corresponds to one row of Table A, R² is n-decyl and R³ is methoxy

### Table 131

Compounds of the formula I.1 in which R¹ for each compound corresponds to one row of Table A, R² is methoxymethyl and R³ is methoxy

### Table 132

Compounds of the formula I.1 in which R¹ for each compound corresponds to one row of Table A, R² is ethoxymethyl and R³ is methoxy

### Table 133

Compounds of the formula I.1 in which R¹ for each compound corresponds to one row of Table A, R² is methyl and R³ is methylthio

### Table 134

Compounds of the formula I.1 in which R¹ for each compound corresponds to one row of Table A, R² is ethyl and R³ is methylthio

### Table 135

Compounds of the formula I.1 in which R¹ for each compound corresponds to one row of Table A, R² is n-propyl and R³ is methylthio

### Table 136

Compounds of the formula I.1 in which R¹ for each compound corresponds to one row of Table A, R² is n-butyl and R³ is methylthio

### Table 137

Compounds of the formula I.1 in which R¹ for each compound corresponds to one row of Table A, R² is n-pentyl and R³ is methylthio

### Table 138

Compounds of the formula I.1 in which R¹ for each compound corresponds to one row of Table A, R² is n-hexyl and R³ is methylthio

### Table 139

Compounds of the formula I.1 in which R¹ for each compound corresponds to one row of Table A, R² is n-heptyl and R³ is methylthio

### Table 140

Compounds of the formula I.1 in which R¹ for each compound corresponds to one row of Table A, R² is n-octyl and R³ is methylthio

### Table 141

Compounds of the formula I.1 in which R¹ for each compound corresponds to one row of Table A, R² is n-nonyl and R³ is methylthio

### Table 142

Compounds of the formula I.1 in which R¹ for each compound corresponds to one row of Table A, R² is n-decyl and R³ is methylthio

### Table 143

Compounds of the formula I.1 in which R¹ for each compound corresponds to one row of Table A, R² is methoxymethyl and R³ is methylthio

### Table 144

Compounds of the formula I.1 in which R¹ for each compound corresponds to one row of Table A, R² is ethoxymethyl and R³ is methylthio

### Table 145

Compounds of the formula I.2 in which R¹ for each compound corresponds to one row of Table A, R² is methyl and R³ is fluorine

### Table 146

Compounds of the formula I.2 in which R¹ for each compound corresponds to one row of Table A, R² is ethyl and R³ is fluorine

### Table 147

Compounds of the formula I.2 in which R¹ for each compound corresponds to one row of Table A, R² is n-propyl and R³ is fluorine

### Table 148

Compounds of the formula I.2 in which R¹ for each compound corresponds to one row of Table A, R² is n-butyl and R³ is fluorine

### Table 149

Compounds of the formula I.2 in which R¹ for each compound corresponds to one row of Table A, R² is n-pentyl and R³ is fluorine

### Table 150

Compounds of the formula I.2 in which R¹ for each compound corresponds to one row of Table A, R² is n-hexyl and R³ is fluorine

### Table 151

Compounds of the formula I.2 in which R¹ for each compound corresponds to one row of Table A, R² is n-heptyl and R³ is fluorine

### Table 152

Compounds of the formula I.2 in which R¹ for each compound corresponds to one row of Table A, R² is n-octyl and R³ is fluorine

### Table 153

Compounds of the formula I.2 in which R¹ for each compound corresponds to one row of Table A, R² is n-nonyl and R³ is fluorine

### Table 154

Compounds of the formula I.2 in which R¹ for each compound corresponds to one row of Table A, R² is n-decyl and R³ is fluorine

### Table 155

Compounds of the formula I.2 in which R¹ for each compound corresponds to one row of Table A, R² is methoxymethyl and R³ is fluorine

### Table 156

Compounds of the formula I.2 in which R¹ for each compound corresponds to one row of Table A, R² is ethoxymethyl and R³ is fluorine

### Table 157

Compounds of the formula I.2 in which R¹ for each compound corresponds to one row of Table A, R² is methyl and R³ is chlorine

### Table 158

Compounds of the formula I.2 in which R¹ for each compound corresponds to one row of Table A, R² is ethyl and R³ is chlorine

### Table 159

Compounds of the formula I.2 in which R¹ for each compound corresponds to one row of Table A, R² is n-propyl and R³ is chlorine

### Table 160

Compounds of the formula I.2 in which R¹ for each compound corresponds to one row of Table A, R² is n-butyl and R³ is chlorine

### Table 161

Compounds of the formula I.2 in which R¹ for each compound corresponds to one row of Table A, R² is n-pentyl and R³ is chlorine

### Table 162

Compounds of the formula I.2 in which R¹ for each compound corresponds to one row of Table A, R² is n-hexyl and R³ is chlorine

### Table 163

Compounds of the formula I.2 in which R¹ for each compound corresponds to one row of Table A, R² is n-heptyl and R³ is chlorine

### Table 164

Compounds of the formula I.2 in which R¹ for each compound corresponds to one row of Table A, R² is n-octyl and R³ is chlorine

### Table 165

Compounds of the formula I.2 in which R¹ for each compound corresponds to one row of Table A, R² is n-nonyl and R³ is chlorine

### Table 166

Compounds of the formula I.2 in which R¹ for each compound corresponds to one row of Table A, R² is n-decyl and R³ is chlorine

### Table 167

Compounds of the formula I.2 in which R¹ for each compound corresponds to one row of Table A, R² is methoxymethyl and R³ is chlorine

### Table 168

Compounds of the formula I.2 in which R¹ for each compound corresponds to one row of Table A, R² is ethoxymethyl and R³ is chlorine

### Table 169

Compounds of the formula I.2 in which R¹ for each compound corresponds to one row of Table A, R² is methyl and R³ is bromine

### Table 170

Compounds of the formula I.2 in which R¹ for each compound corresponds to one row of Table A, R² is ethyl and R³ is bromine

### Table 171

Compounds of the formula I.2 in which R¹ for each compound corresponds to one row of Table A, R² is n-propyl and R³ is bromine

### Table 172

Compounds of the formula I.2 in which R¹ for each compound corresponds to one row of Table A, R² is n-butyl and R³ is bromine

### Table 173

Compounds of the formula I.2 in which R¹ for each compound corresponds to one row of Table A, R² is n-pentyl and R³ is bromine

### Table 174

Compounds of the formula I.2 in which R¹ for each compound corresponds to one row of Table A, R² is n-hexyl and R³ is bromine

### Table 175

Compounds of the formula I.2 in which R¹ for each compound corresponds to one row of Table A, R² is n-heptyl and R³ is bromine

### Table 176

Compounds of the formula I.2 in which R¹ for each compound corresponds to one row of Table A, R² is n-octyl and R³ is bromine

### Table 177

Compounds of the formula I.2 in which R¹ for each compound corresponds to one row of Table A, R² is n-nonyl and R³ is bromine

### Table 178

Compounds of the formula I.2 in which R¹ for each compound corresponds to one row of Table A, R² is n-decyl and R³ is bromine

### Table 179

Compounds of the formula I.2 in which R¹ for each compound corresponds to one row of Table A, R² is methoxymethyl and R³ is bromine

### Table 180

Compounds of the formula I.2 in which R¹ for each compound corresponds to one row of Table A, R² is ethoxymethyl and R³ is bromine

### Table 181

Compounds of the formula I.2 in which R¹ for each compound corresponds to one row of Table A, R² is methyl and R³ is cyano

### Table 182

Compounds of the formula I.2 in which R¹ for each compound corresponds to one row of Table A, R² is ethyl and R³ is cyano

### Table 183

Compounds of the formula I.2 in which R¹ for each compound corresponds to one row of Table A, R² is n-propyl and R³ is cyano

### Table 184

Compounds of the formula I.2 in which R¹ for each compound corresponds to one row of Table A, R² is n-butyl and R³ is cyano

### Table 185

Compounds of the formula I.2 in which R¹ for each compound corresponds to one row of Table A, R² is n-pentyl and R³ is cyano

### Table 186

Compounds of the formula I.2 in which R¹ for each compound corresponds to one row of Table A, R² is n-hexyl and R³ is cyano

### Table 187

Compounds of the formula I.2 in which R¹ for each compound corresponds to one row of Table A, R² is n-heptyl and R³ is cyano

### Table 188

Compounds of the formula I.2 in which R¹ for each compound corresponds to one row of Table A, R² is n-octyl and R³ is cyano

### Table 189

Compounds of the formula I.2 in which R¹ for each compound corresponds to one row of Table A, R² is n-nonyl and R³ is cyano

### Table 190

Compounds of the formula I.2 in which R¹ for each compound corresponds to one row of Table A, R² is n-decyl and R³ is cyano

### Table 191

Compounds of the formula I.2 in which R¹ for each compound corresponds to one row of Table A, R² is methoxymethyl and R³ is cyano

### Table 192

Compounds of the formula I.2 in which R¹ for each compound corresponds to one row of Table A, R² is ethoxymethyl and R³ is cyano

### Table 193

Compounds of the formula I.2 in which R¹ for each compound corresponds to one row of Table A, R² is methyl and R³ is hydroxyl

### Table 194

Compounds of the formula I.2 in which R¹ for each compound corresponds to one row of Table A, R² is ethyl and R³ is hydroxyl

### Table 195

Compounds of the formula I.2 in which R¹ for each compound corresponds to one row of Table A, R² is n-propyl and R³ is hydroxyl

### Table 196

Compounds of the formula I.2 in which R¹ for each compound corresponds to one row of Table A, R² is n-butyl and R³ is hydroxyl

### Table 197

Compounds of the formula I.2 in which R¹ for each compound corresponds to one row of Table A, R² is n-pentyl and R³ is hydroxyl

### Table 198

Compounds of the formula I.2 in which R¹ for each compound corresponds to one row of Table A, R² is n-hexyl and R³ is hydroxyl

### Table 199

Compounds of the formula I.2 in which R¹ for each compound corresponds to one row of Table A, R² is n-heptyl and R³ is hydroxyl

### Table 200

Compounds of the formula I.2 in which R¹ for each compound corresponds to one row of Table A, R² is n-octyl and R³ is hydroxyl

### Table 201

Compounds of the formula I.2 in which R¹ for each compound corresponds to one row of Table A, R² is n-nonyl and R³ is hydroxyl

### Table 202

Compounds of the formula I.2 in which R¹ for each compound corresponds to one row of Table A, R² is n-decyl and R³ is hydroxyl

### Table 203

Compounds of the formula I.2 in which R¹ for each compound corresponds to one row of Table A, R² is methoxymethyl and R³ is hydroxyl

### Table 204

Compounds of the formula I.2 in which R¹ for each compound corresponds to one row of Table A, R² is ethoxymethyl and R³ is hydroxyl

### Table 205

Compounds of the formula I.2 in which R¹ for each compound corresponds to one row of Table A, R² is methyl and R³ is mercapto

### Table 206

Compounds of the formula I.2 in which R¹ for each compound corresponds to one row of Table A, R² is ethyl and R³ is mercapto

### Table 207

Compounds of the formula I.2 in which R¹ for each compound corresponds to one row of Table A, R² is n-propyl and R³ is mercapto

### Table 208

Compounds of the formula I.2 in which R¹ for each compound corresponds to one row of Table A, R² is n-butyl and R³ is mercapto

### Table 209

Compounds of the formula I.2 in which R¹ for each compound corresponds to one row of Table A, R² is n-pentyl and R³ is mercapto

### Table 210

Compounds of the formula I.2 in which R¹ for each compound corresponds to one row of Table A, R² is n-hexyl and R³ is mercapto

### Table 211

Compounds of the formula I.2 in which R¹ for each compound corresponds to one row of Table A, R² is n-heptyl and R³ is mercapto

### Table 212

Compounds of the formula I.2 in which R¹ for each compound corresponds to one row of Table A, R² is n-octyl and R³ is mercapto

### Table 213

Compounds of the formula I.2 in which R¹ for each compound corresponds to one row of Table A, R² is n-nonyl and R³ is mercapto

### Table 214

Compounds of the formula I.2 in which R¹ for each compound corresponds to one row of Table A, R² is n-decyl and R³ is mercapto

### Table 215

Compounds of the formula I.2 in which R¹ for each compound corresponds to one row of Table A, R² is methoxymethyl and R³ is mercapto

### Table 216

Compounds of the formula I.2 in which R¹ for each compound corresponds to one row of Table A, R² is ethoxymethyl and R³ is mercapto

### Table 217

Compounds of the formula I.2 in which R¹ for each compound corresponds to one row of Table A, R² is methyl and R³ is amino

### Table 218

Compounds of the formula I.2 in which R¹ for each compound corresponds to one row of Table A, R² is ethyl and R³ is amino

### Table 219

Compounds of the formula I.2 in which R¹ for each compound corresponds to one row of Table A, R² is n-propyl and R³ is amino

### Table 220

Compounds of the formula I.2 in which R¹ for each compound corresponds to one row of Table A, R² is n-butyl and R³ is amino

### Table 221

Compounds of the formula I.2 in which R¹ for each compound corresponds to one row of Table A, R² is n-pentyl and R³ is amino

### Table 222

Compounds of the formula I.2 in which R¹ for each compound corresponds to one row of Table A, R² is n-hexyl and R³ is amino

### Table 223

Compounds of the formula I.2 in which R¹ for each compound corresponds to one row of Table A, R² is n-heptyl and R³ is amino

### Table 224

Compounds of the formula I.2 in which R¹ for each compound corresponds to one row of Table A, R² is n-octyl and R³ is amino

### Table 225

Compounds of the formula I.2 in which R¹ for each compound corresponds to one row of Table A, R² is n-nonyl and R³ is amino

### Table 226

Compounds of the formula I.2 in which R¹ for each compound corresponds to one row of Table A, R² is n-decyl and R³ is amino

### Table 227

Compounds of the formula I.2 in which R¹ for each compound corresponds to one row of Table A, R² is methoxymethyl and R³ is amino

### Table 228

Compounds of the formula I.2 in which R¹ for each compound corresponds to one row of Table A, R² is ethoxymethyl and R³ is amino

### Table 229

Compounds of the formula I.2 in which R¹ for each compound corresponds to one row of Table A, R² is methyl and R³ is trifluoromethyl

### Table 230

Compounds of the formula I.2 in which R¹ for each compound corresponds to one row of Table A, R² is ethyl and R³ is trifluoromethyl

### Table 231

Compounds of the formula I.2 in which R¹ for each compound corresponds to one row of Table A, R² is n-propyl and R³ is trifluoromethyl

### Table 232

Compounds of the formula I.2 in which R¹ for each compound corresponds to one row of Table A, R² is n-butyl and R³ is trifluoromethyl

### Table 233

Compounds of the formula I.2 in which R¹ for each compound corresponds to one row of Table A, R² is n-pentyl and R³ is trifluoromethyl

### Table 234

Compounds of the formula I.2 in which R¹ for each compound corresponds to one row of Table A, R² is n-hexyl and R³ is trifluoromethyl

### Table 235

Compounds of the formula I.2 in which R¹ for each compound corresponds to one row of Table A, R² is n-heptyl and R³ is trifluoromethyl

### Table 236

Compounds of the formula I.2 in which R¹ for each compound corresponds to one row of Table A, R² is n-octyl and R³ is trifluoromethyl

### Table 237

Compounds of the formula I.2 in which R¹ for each compound corresponds to one row of Table A, R² is n-nonyl and R³ is trifluoromethyl

### Table 238

Compounds of the formula I.2 in which R¹ for each compound corresponds to one row of Table A, R² is n-decyl and R³ is trifluoromethyl

### Table 239

Compounds of the formula I.2 in which R¹ for each compound corresponds to one row of Table A, R² is methoxymethyl and R³ is trifluoromethyl

### Table 240

Compounds of the formula I.2 in which R¹ for each compound corresponds to one row of Table A, R² is ethoxymethyl and R³ is trifluoromethyl

### Table 241

Compounds of the formula I.2 in which R¹ for each compound corresponds to one row of Table A, R² is methyl and R³ is ethyl

### Table 242

Compounds of the formula I.2 in which R¹ for each compound corresponds to one row of Table A, R² is ethyl and R³ is ethyl

### Table 243

Compounds of the formula I.2 in which R¹ for each compound corresponds to one row of Table A, R² is n-propyl and R³ is ethyl

### Table 244

Compounds of the formula I.2 in which R¹ for each compound corresponds to one row of Table A, R² is n-butyl and R³ is ethyl

### Table 245

Compounds of the formula I.2 in which R¹ for each compound corresponds to one row of Table A, R² is n-pentyl and R³ is ethyl

### Table 246

Compounds of the formula I.2 in which R¹ for each compound corresponds to one row of Table A, R² is n-hexyl and R³ is ethyl

### Table 247

Compounds of the formula I.2 in which R¹ for each compound corresponds to one row of Table A, R² is n-heptyl and R³ is ethyl

### Table 248

Compounds of the formula I.2 in which R¹ for each compound corresponds to one row of Table A, R² is n-octyl and R³ is ethyl

### Table 249

Compounds of the formula I.2 in which R¹ for each compound corresponds to one row of Table A, R² is n-nonyl and R³ is ethyl

### Table 250

Compounds of the formula I.2 in which R¹ for each compound corresponds to one row of Table A, R² is n-decyl and R³ is ethyl

### Table 251

Compounds of the formula I.2 in which R¹ for each compound corresponds to one row of Table A, R² is methoxymethyl and R³ is ethyl

### Table 252

Compounds of the formula I.2 in which R¹ for each compound corresponds to one row of Table A, R² is ethoxymethyl and R³ is ethyl

### Table 253

Compounds of the formula I.2 in which R¹ for each compound corresponds to one row of Table A, R² is methyl and R³ is n-propyl

### Table 254

Compounds of the formula I.2 in which R¹ for each compound corresponds to one row of Table A, R² is ethyl and R³ is n-propyl

### Table 255

Compounds of the formula I.2 in which R¹ for each compound corresponds to one row of Table A, R² is n-propyl and R³ is n-propyl.

### Table 256

Compounds of the formula I.2 in which R¹ for each compound corresponds to one row of Table A, R² is n-butyl and R³ is n-propyl

### Table 257

Compounds of the formula I.2 in which R¹ for each compound corresponds to one row of Table A, R² is n-pentyl and R³ is n-propyl

### Table 258

Compounds of the formula I.2 in which R¹ for each compound corresponds to one row of Table A, R² is n-hexyl and R³ is n-propyl

### Table 259

Compounds of the formula I.2 in which R¹ for each compound corresponds to one row of Table A, R² is n-heptyl and R³ is n-propyl

### Table 260

Compounds of the formula I.2 in which R¹ for each compound corresponds to one row of Table A, R² is n-octyl and R³ is n-propyl

### Table 261

Compounds of the formula I.2 in which R¹ for each compound corresponds to one row of Table A, R² is n-nonyl and R³ is n-propyl

### Table 262

Compounds of the formula I.2 in which R¹ for each compound corresponds to one row of Table A, R² is n-decyl and R³ is n-propyl

### Table 263

Compounds of the formula I.2 in which R¹ for each compound corresponds to one row of Table A, R² is methoxymethyl and R³ is n-propyl

### Table 264

Compounds of the formula I.2 in which R¹ for each compound corresponds to one row of Table A, R² is ethoxymethyl and R³ is n-propyl

### Table 265

Compounds of the formula I.2 in which R¹ for each compound corresponds to one row of Table A, R² is methyl and R³ is methoxy

### Table 266

Compounds of the formula I.2 in which R¹ for each compound corresponds to one row of Table A, R² is ethyl and R³ is methoxy

### Table 267

Compounds of the formula I.2 in which R¹ for each compound corresponds to one row of Table A, R² is n-propyl and R³ is methoxy

### Table 268

Compounds of the formula I.2 in which R¹ for each compound corresponds to one row of Table A, R² is n-butyl and R³ is methoxy

### Table 269

Compounds of the formula I.2 in which R¹ for each compound corresponds to one row of Table A, R² is n-pentyl and R³ is methoxy

### Table 270

Compounds of the formula I.2 in which R¹ for each compound corresponds to one row of Table A, R² is n-hexyl and R³ is methoxy

### Table 271

Compounds of the formula I.2 in which R¹ for each compound corresponds to one row of Table A, R² is n-heptyl and R³ is methoxy

### Table 272

Compounds of the formula I.2 in which R¹ for each compound corresponds to one row of Table A, R² is n-octyl and R³ is methoxy

### Table 273

Compounds of the formula I.2 in which R¹ for each compound corresponds to one row of Table A, R² is n-nonyl and R³ is methoxy

### Table 274

Compounds of the formula I.2 in which R¹ for each compound corresponds to one row of Table A, R² is n-decyl and R³ is methoxy

### Table 275

Compounds of the formula I.2 in which R¹ for each compound corresponds to one row of Table A, R² is methoxymethyl and R³ is methoxy

### Table 276

Compounds of the formula I.2 in which R¹ for each compound corresponds to one row of Table A, R² is ethoxymethyl and R³ is methoxy

### Table 277

Compounds of the formula I.2 in which R¹ for each compound corresponds to one row of Table A, R² is methyl and R³ is methylthio

### Table 278

Compounds of the formula I.2 in which R¹ for each compound corresponds to one row of Table A, R² is ethyl and R³ is methylthio

### Table 279

Compounds of the formula I.2 in which R¹ for each compound corresponds to one row of Table A, R² is n-propyl and R³ is methylthio

### Table 280

Compounds of the formula I.2 in which R¹ for each compound corresponds to one row of Table A, R² is n-butyl and R³ is methylthio

### Table 281

Compounds of the formula I.2 in which R¹ for each compound corresponds to one row of Table A, R² is n-pentyl and R³ is methylthio

### Table 282

Compounds of the formula I.2 in which R¹ for each compound corresponds to one row of Table A, R² is n-hexyl and R³ is methylthio

### Table 283

Compounds of the formula I.2 in which R¹ for each compound corresponds to one row of Table A, R² is n-heptyl and R³ is methylthio

### Table 284

Compounds of the formula I.2 in which R¹ for each compound corresponds to one row of Table A, R² is n-octyl and R³ is methylthio

### Table 285

Compounds of the formula I.2 in which R¹ for each compound corresponds to one row of Table A, R² is n-nonyl and R³ is methylthio

### Table 286

Compounds of the formula I.2 in which R¹ for each compound corresponds to one row of Table A, R² is n-decyl and R³ is methylthio

### Table 287

Compounds of the formula I.2 in which R¹ for each compound corresponds to one row of Table A, R² is methoxymethyl and R³ is methylthio

### Table 288

Compounds of the formula I.2 in which R¹ for each compound corresponds to one row of Table A, R² is ethoxymethyl and R³ is methylthio

TABLE A

| No. | R¹ |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH₂CH₂CH₃ |
| A-6 | CH(CH₃)CH₂CH₃ |
| A-7 | CH₂CH(CH₃)₂ |
| A-8 | C(CH₃)₃ |
| A-9 | CH₂CH₂CH₂CH₂CH₃ |
| A-10 | CH(CH₃)CH₂CH₂CH₃ |
| A-11 | CH₂CH(CH₃)CH₂CH₃ |
| A-12 | CH₂CH₂CH(CH₃)CH₃ |
| A-13 | CH₂CH₂CH(CH₃)₂ |
| A-14 | CH(CH₃)CH(CH₃)CH₃ |
| A-15 | CH(CH₃)CH(CH₃)₂ |
| A-16 | CH₂C(CH₃)₃ |
| A-17 | CH₂CH₂CH₂CH₂CH₂CH₃ |
| A-18 | CH(CH₃)CH₂CH₂CH₂CH₃ |
| A-19 | CH₂CH(CH₃)CH₂CH₂CH₃ |
| A-20 | CH₂CH₂CH(CH₃)CH₂CH₃ |
| A-21 | CH₂CH₂CH(CH₃)₂CH₂ |
| A-22 | CH₂CH₂CH₂CH(CH₃)₂ |
| A-23 | CH(CH₃)CH(CH₃)CH₂CH₃ |
| A-24 | CH(CH₃)CH₂CH(CH₃)₂ |
| A-25 | CH₂CH₂C(CH₃)₃ |
| A-26 | CH(CH₃)CH₂CH(CH₃)CH₃ |
| A-27 | CH₂CH₂CH₂CH₂CH₂CH₂CH₃ |
| A-28 | CH(CH₃)CH₂CH₂CH₂CH₂CH₃ |
| A-29 | CH₂CH(CH₃)CH₂CH₂CH₂CH₃ |
| A-30 | CH₂CH₂CH(CH₃)CH₂CH₂CH₃ |
| A-31 | CH₂CH₂CH₂CH(CH₃)CH₂CH₃ |
| A-32 | CH₂CH₂CH₂CH₂CH(CH₃)CH₃ |
| A-33 | CH₂CH₂CH₂CH₂CH(CH₃)₂ |
| A-34 | CH(CH₃)CH(CH₃)CH₂CH₂CH₃ |
| A-35 | CH₂CH(CH₃)CH(CH₃)CH₂CH₃ |
| A-36 | CH₂CH₂CH₂C(CH₃)₃ |
| A-37 | CH(CH₃)CH₂CH(CH₃)CH₂CH₃ |
| A-38 | CH₂CH(CH₃)CH(CH₃)CH₂CH₃ |
| A-39 | CH(CH₃)CH₂CH₂CH(CH₃)CH₃ |
| A-40 | CH₂CH₂CH₂CH₂CH₂CH₂CH₂CH₃ |
| A-41 | CH(CH₃)CH₂CH₂CH₂CH₂CH₂CH₃ |
| A-42 | CH₂CH(CH₃)CH₂CH₂CH₂CH₂CH₃ |
| A-43 | CH₂CH₂CH(CH₃)CH₂CH₂CH₂CH₃ |
| A-44 | CH₂CH₂CH₂CH(CH₃)CH₂CH₂CH₃ |
| A-45 | CH₂CH₂CH₂CH₂CH(CH₃)CH₂CH₃ |
| A-46 | CH₂CH₂CH₂CH₂CH₂CH(CH₃)₂ |
| A-47 | CH₂CH₂CH₂CH₂C(CH₃)₃ |
| A-48 | CH(CH₃)CH(CH₃)CH₂CH₂CH₂CH₃ |
| A-49 | CH₂CH(CH₃)CH(CH₃)CH₂CH₂CH₃ |
| A-50 | CH₂CH₂CH₂C(CH₃)₂CH₂CH₃ |
| A-51 | CH(CH₃)CH₂CH(CH₃)CH₂CH₂CH₃ |
| A-52 | CH₂CH(CH₃)CH(CH₃)CH₂CH₂CH₃ |
| A-53 | CH(CH₃)CH₂CH₂CH(CH₃)CH₂CH₃ |
| A-54 | CH(CH₃)CH₂CH₂CH₂CH(CH₃)₂ |
| A-55 | CH₂CH₂CH(CH₃)CH₂C(CH₃)₃ |
| A-56 | CH₂CH₂CH₂CH₂CH₂CH₂CH₂CH₂CH₃ |
| A-57 | CH(CH₃)CH₂CH₂CH₂CH₂CH₂CH₂CH₃ |
| A-58 | CH₂CH(CH₃)CH₂CH₂CH₂CH₂CH₂CH₃ |
| A-59 | CH₂CH₂CH(CH₃)CH₂CH₂CH₂CH₂CH₃ |
| A-60 | CH₂CH₂CH₂CH(CH₃)CH₂CH₂CH₂CH₃ |
| A-61 | CH₂CH₂CH₂CH₂CH(CH₃)CH₂CH₂CH₃ |
| A-62 | CH₂CH₂CH₂CH₂CH₂CH₂C(CH₃)₃ |
| A-63 | CH(CH₃)CH(CH₃)CH₂CH₂CH₂CH₂CH₃ |
| A-64 | CH₂CH(CH₃)CH(CH₃)CH₂CH₂CH₂CH₃ |
| A-65 | CH₂CH₂CH₂C(CH₃)₂CH₂CH₂CH₃ |
| A-66 | CH(CH₃)CH₂CH(CH₃)CH₂CH₂CH₂CH₃ |
| A-67 | CH₂CH(CH₃)CH(CH₃)CH₂CH₂CH₂CH₃ |
| A-68 | CH(CH₃)CH₂CH₂CH(CH₃)CH₂CH₂CH₃ |
| A-69 | CH(CH₃)CH₂CH₂CH₂C(CH₃)₃ |
| A-70 | CH₂CH(CH₃)CH₂CH₂CH(CH₃)₃ |
| A-71 | CH(CH₃)CH₂CH₂CH₂CH₂CH(CH₃)₂ |
| A-72 | CH₂CH(CH₃)CH₂CH₂CH₂CH(CH₃)₂ |
| A-73 | CH₂CH₂CH₂CH₂CH₂CH₂CH₂CH₂CH₂CH₃ |
| A-74 | CH(CH₃)CH₂CH₂CH₂CH₂CH₂CH₂CH₂CH₃ |
| A-75 | CH₂CH(CH₃)CH₂CH₂CH₂CH₂CH₂CH₂CH₃ |
| A-76 | CH₂CH₂CH(CH₃)CH₂CH₂CH₂CH₂CH₂CH₃ |
| A-77 | CH₂CH₂CH(CH₃)CH₂CH₂CH₂CH₂CH₂ |
| A-78 | CH₂CH₂CH₂CH(CH₃)CH₂CH₂CH₂CH₃ |
| A-79 | CH₂CH₂CH₂CH₂CH₂CH₂C(CH₃)₃ |
| A-80 | CH(CH₃)CH(CH₃)CH₂CH₂CH₂CH₂CH₂CH₃ |
| A-81 | CH₂CH(CH₃)CH(CH₃)CH₂CH₂CH₂CH₂CH₃ |
| A-82 | CH₂CH₂CH₂C(CH₃)₂CH₂CH₂CH₂CH₃ |
| A-83 | CH(CH₃)CH₂CH(CH₃)CH₂CH₂CH₂CH₂CH₃ |
| A-84 | CH₂CH(CH₃)CH(CH₃)CH₂CH₂CH₂CH₂CH₃ |
| A-85 | CH(CH₃)CH₂CH₂CH(CH₃)CH₂CH₂CH₂CH₃ |
| A-86 | CH(CH₃)CH₂CH₂CH₂CH(CH₃)CH₂CH₂CH₃ |
| A-87 | CH(CH₃)CH₂CH₂CH₂CH₂CH(CH₃)CH₂CH₃ |
| A-88 | CH(CH₃)CH₂CH₂CH₂CH₂CH₂CH(CH₃)₂ |
| A-89 | CH(CH₃)CH₂CH₂CH₂CH₂CH₂C(CH₃)CH₃ |
| A-90 | CH₂CH(CH₃)CH₂CH₂CH₂CH₂CH(CH₃)CH₃ |
| A-91 | CH(CH₃)CH₂CH₂CH₂CH₂C(CH₃)₃ |
| A-92 | CH₂CH(CH₃)CH₂CH₂CH₂C(CH₃)₃ |
| A-93 | CH₂CH₂CH₂CH₂CH₂CH₂CH₂CH₂CH₂CH₂CH₃ |
| A-94 | CH(CH₃)CH₂CH₂CH₂CH₂CH₂CH₂CH₂CH₂CH₃ |
| A-95 | CH₂CH(CH₃)CH₂CH₂CH₂CH₂CH₂CH₂CH₂CH₃ |
| A-96 | CH₂CH₂CH(CH₃)CH₂CH₂CH₂CH₂CH₂CH₂CH₃ |
| A-97 | CH₂CH₂CH₂CH(CH₃)CH₂CH₂CH₂CH₂CH₂CH₃ |
| A-98 | CH₂CH₂CH₂CH₂CH(CH₃)CH₂CH₂CH₂CH₂CH₃ |
| A-99 | CH₂CH₂CH₂CH₂CH₂CH₂CH₂C(CH₃)₃ |
| A-100 | CH(CH₃)CH(CH₃)CH₂CH₂CH₂CH₂CH₂CH₂CH₃ |
| A-101 | CH₂CH(CH₃)CH(CH₃)CH₂CH₂CH₂CH₂CH₂CH₃ |
| A-102 | CH₂CH₂CH₂C(CH₃)₂CH₂CH₂CH₂CH₂CH₃ |
| A-103 | CH(CH₃)CH₂CH(CH₃)CH₂CH₂CH₂CH₂CH₂CH₃ |
| A-104 | CH₂CH(CH₃)CH(CH₃)CH₂CH₂CH₂CH₂CH₂CH₃ |
| A-105 | CH(CH₃)CH₂CH₂CH(CH₃)CH₂CH₂CH₂CH₂CH₃ |
| A-106 | CH(CH₃)CH₂CH₂CH₂CH(CH₃)CH₂CH₂CH₂CH₃ |
| A-107 | CH(CH₃)CH₂CH₂CH₂CH₂CH(CH₃)CH₂CH₂CH₃ |
| A-108 | CH(CH₃)CH₂CH₂CH₂CH₂CH₂CH(CH₃)CH₂CH₃ |
| A-109 | CH(CH₃)CH₂CH₂CH₂CH₂CH₂CH₂CH(CH₃)₂ |
| A-110 | CH₂CH(CH₃)CH₂CH₂CH₂CH₂CH(CH₃)CH₂CH₃ |
| A-111 | CH₂CH(CH₃)CH₂CH₂CH₂CH₂CH(CH₃)CH₂CH₃ |
| A-112 | CH₂CH₂CH(CH₃)CH₂CH₂CH₂CH₂CH(CH₃)₂ |
| A-113 | CH₂CH(CH₃)CH₂CH₂CH₂CH₂C(CH₃)₃ |
| A-114 | CH₂CH₂CH₂CH₂CH₂CH₂CH₂CH₂CH₂CH₂CH₂CH₃ |
| A-115 | CH(CH₃)CH₂CH₂CH₂CH₂CH₂CH₂CH₂CH₂CH₂CH₃ |
| A-116 | CH₂CH(CH₃)CH₂CH₂CH₂CH₂CH₂CH₂CH₂CH₂CH₃ |
| A-117 | CH₂CH₂CH(CH₃)CH₂CH₂CH₂CH₂CH₂CH₂CH₂CH₃ |
| A-118 | CH₂CH₂CH₂CH(CH₃)CH₂CH₂CH₂CH₂CH₂CH₂CH₂ |
| A-119 | CH₂CH₂CH₂CH₂CH(CH₃)CH₂CH₂CH₂CH₂CH₂CH₃ |
| A-120 | CH₂CH₂CH₂CH₂CH₂CH(CH₃)CH₂CH₂CH₂CH₂CH₃ |
| A-121 | CH₂CH₂CH₂CH₂CH₂CH₂CH(CH₃)CH₂CH₂CH₂CH₃ |
| A-122 | CH₂CH₂CH₂CH₂CH₂CH₂CH₂CH(CH₃)CH₂CH₂CH₃ |
| A-123 | CH₂CH₂CH₂CH₂CH₂CH₂CH₂CH₂CH(CH₃)CH₂CH₃ |
| A-124 | CH₂CH₂CH₂CH₂CH₂CH₂CH₂CH₂CH₂CH(CH₃)₂ |
| A-125 | CH(CH₃)CH(CH₃)CH₂CH₂CH₂CH₂CH₂CH₂CH₂CH₃ |
| A-126 | CH₂CH(CH₃)CH(CH₃)CH₂CH₂CH₂CH₂CH₂CH₂CH₃ |
| A-127 | CH₂CH₂CH₂C(CH₃)₂CH₂CH₂CH₂CH₂CH₂CH₃ |
| A-128 | CH₂CH₂CH₂CH₂CH(CH₃)CH₂CH₂CH₂CH₂CH₂CH₃ |
| A-129 | CH(CH₃)CH₂CH(CH₃)CH₂CH₂CH₂CH₂CH₂CH₂CH₃ |
| A-130 | CH(CH₃)CH₂CH₂CH(CH₃)CH₂CH₂CH₂CH₂CH₂CH₃ |
| A-131 | CH(CH₃)CH₂CH₂CH₂CH(CH₃)CH₂CH₂CH₂CH₂CH₃ |
| A-132 | CH(CH₃)CH₂CH₂CH₂CH₂CH(CH₃)CH₂CH₂CH₂CH₃ |
| A-133 | CH(CH₃)CH₂CH₂CH₂CH₂CH₂CH(CH₃)CH₂CH₂CH₃ |
| A-134 | CH(CH₃)CH₂CH₂CH₂CH₂CH₂CH₂CH(CH₃)CH₂CH₃ |
| A-135 | CH₂CH(CH₃)CH₂CH₂CH₂CH₂CH(CH₃)CH₂CH₂CH₃ |
| A-136 | CH₂CH₂CH(CH₃)CH₂CH₂CH₂CH₂CH(CH₃)CH₂CH₃ |
| A-137 | CH₂CH₂CH₂CH(CH₃)CH₂CH₂CH₂CH(CH₃)CH₂CH₃ |
| A-138 | CH₂CH(CH₃)CH₂CH₂CH₂CH₂CH₂C(CH₃)₃ |
| A-139 | CH₂CH₂CH₂—O—CH₃ |
| A-140 | CH₂CH₂CH₂—O—CH₂CH₃ |
| A-141 | CH₂CH₂CH₂—O—CH₂CH₂CH₃ |
| A-142 | CH₂CH₂CH₂—O—CH₂CH₂CH₂CH₃ |
| A-143 | CH₂CH₂CH₂—O—CH₂CH₂CH₂CH₂CH₃ |
| A-144 | CH₂CH₂CH₂—O—CH₂CH₂CH₂CH₂CH₂CH₃ |
| A-145 | CH₂CH₂CH₂—O—CH₂CH₂CH₂CH₂CH₂CH₂CH₃ |
| A-146 | CH₂CH₂CH₂—O—CH₂CH₂CH₂CH₂CH₂CH₂CH₂CH₃ |
| A-147 | CH₂CH₂CH₂—O—CH₂CH₂CH₂CH₂CH₂CH₂CH₂CH₂CH₃ |
| A-148 | CH₂CH₂CH₂—O—CH(CH₃)₂ |
| A-149 | CH₂CH₂CH₂—O—C(CH₃)₃ |
| A-150 | CH₂CH₂CH₂—O—CH₂C(CH₃)₃ |
| A-151 | CH₂CH₂CH₂—O—CH(CH₃)CH₂C(CH₃)₃ |
| A-152 | CH₂CH₂CH₂—O—CH(CH₂CH₃)CH₂C(CH₃)₃ |
| A-153 | CH₂CH₂CH₂—O—CH₂CH(CH₃)CH₂CH(CH₃)₂ |
| A-154 | CH₂CH₂CH₂—O—CH₂CH(CH₂CH₃)CH₂CH₂CH₃ |
| A-155 | CH₂CH₂CH₂—O—CH₂CH₂CH(CH₃)CH₂CH(CH₃)₂ |
| A-156 | CH₂CH₂CH₂—O—CH₂CH₂CH(CH₃)CH₂C(CH₃)₃ |
| A-157 | CH₂CH₂CH₂—O—CH₂CH₂CH(CH₃)CH₂CH₂CH(CH₃)₂ |
| A-158 | CH₂CH₂CH₂—O—CH₂CH₂CH(CH₃)CH₂CH₂CH₂CH(CH₃)₂ |
| A-159 | CH₂CH₂CH₂CH₂—O—CH₃ |
| A-160 | CH₂CH₂CH₂CH₂—O—CH₂CH₃ |
| A-161 | CH₂CH₂CH₂CH₂—O—CH₂CH₂CH₃ |
| A-162 | CH₂CH₂CH₂CH₂—O—CH₂CH₂CH₂CH₃ |
| A-163 | CH₂CH₂CH₂CH₂—O—CH₂CH₂CH₂CH₂CH₃ |
| A-164 | CH₂CH₂CH₂CH₂—O—CH₂CH₂CH₂CH₂CH₂CH₃ |
| A-165 | CH₂CH₂CH₂CH₂—O—CH₂CH₂CH₂CH₂CH₂CH₂CH₃ |
| A-166 | CH₂CH₂CH₂CH₂—O—CH₂CH₂CH₂CH₂CH₂CH₂CH₂CH₃ |
| A-167 | CH₂CH₂CH₂CH₂—O—CH(CH₃)₂ |
| A-168 | CH₂CH₂CH₂CH₂—O—C(CH₃)₃ |
| A-169 | CH₂CH₂CH₂CH₂—O—CH₂C(CH₃)₃ |
| A-170 | CH₂CH₂CH₂CH₂—O—CH(CH₃)CH₂C(CH₃)₃ |
| A-171 | CH₂CH₂CH₂CH₂—O—CH(CH₂CH₃)CH₂C(CH₃)₃ |
| A-172 | CH₂CH₂CH₂CH₂—O—CH₂CH(CH₃)CH₂CH(CH₃)₂ |
| A-173 | CH₂CH₂CH₂CH₂—O—CH₂CH(CH₂CH₃)CH₂CH₂CH₃ |
| A-174 | CH₂CH₂CH₂CH₂—O—CH₂CH₂CH(CH₃)CH₂CH(CH₃)₂ |
| A-175 | CH₂CH₂CH₂CH₂—O—CH₂CH₂CH(CH₃)CH₂C(CH₃)₃ |
| A-176 | CH₂CH₂CH₂CH₂—O—CH₂CH₂CH(CH₃)CH₂CH₂CH(CH₃)₂ |
| A-177 | CH₂CH₂CH₂CH₂—O— |
| | CH₂CH₂CH(CH₃)CH₂CH₂CH₂CH(CH₃)₂ |
| A-178 | CH₂CH₂CH₂CH₂CH₂—O—CH₃ |
| A-179 | CH₂CH₂CH₂CH₂CH₂—O—CH₂CH₃ |
| A-180 | CH₂CH₂CH₂CH₂CH₂—O—CH₂CH₂CH₃ |
| A-181 | CH₂CH₂CH₂CH₂CH₂—O—CH₂CH₂CH₂CH₃ |
| A-182 | CH₂CH₂CH₂CH₂CH₂—O—CH₂CH₂CH₂CH₂CH₃ |
| A-183 | CH₂CH₂CH₂CH₂CH₂—O—CH₂CH₂CH₂CH₂CH₂CH₃ |
| A-184 | CH₂CH₂CH₂CH₂CH₂—O—CH₂CH₂CH₂CH₂CH₂CH₂CH₃ |
| A-185 | CH₂CH₂CH₂CH₂CH₂—O—CH₂CH₂CH₂CH₂CH₂CH₂CH₂CH₃ |
| A-186 | CH₂CH₂CH₂CH₂CH₂—O—CH(CH₃)₂ |
| A-187 | CH₂CH₂CH₂CH₂CH₂—O—C(CH₃)₃ |
| A-188 | CH₂CH₂CH₂CH₂CH₂—O—CH₂C(CH₃)₃ |
| A-189 | CH₂CH₂CH₂CH₂CH₂—O—CH(CH₃)CH₂C(CH₃)₃ |
| A-190 | CH₂CH₂CH₂CH₂CH₂—O—CH(CH₂CH₃)CH₂C(CH₃)₃ |
| A-191 | CH₂CH₂CH₂CH₂CH₂—O—CH₂CH(CH₃)CH₂CH(CH₃)₂ |
| A-192 | CH₂CH₂CH₂CH₂CH₂—O—CH₂CH(CH₂CH₃)CH₂CH₂CH₃ |
| A-193 | CH₂CH₂CH₂CH₂CH₂— |
| | O—CH₂CH₂CH(CH₃)CH₂CH₂CH(CH₃)₂ |
| A-194 | CH₂CH₂CH₂CH₂CH₂—O—CH₂CH₂CH(CH₃)CH₂CH(CH₃)₂ |
| A-195 | CH₂CH₂CH₂CH₂CH₂—O—CH₂CH₂CH(CH₃)CH₂C(CH₃)₃ |
| A-196 | CH₂F |
| A-197 | CH₂Cl |
| A-198 | CH₂Br |
| A-199 | CHF₂ |
| A-200 | CHCl₂ |
| A-201 | CF₃ |
| A-202 | CCl₃ |
| A-203 | CHFCH₃ |
| A-204 | CHClCH₃ |
| A-205 | CH₂CH₂F |
| A-206 | CH₂CH₂Cl |
| A-207 | CH₂CH₂Br |
| A-208 | CCl₂CH₃ |
| A-209 | CF₂CH₃ |
| A-210 | CH₂CHF₂ |
| A-211 | CH₂CHCl₂ |
| A-212 | CH₂CF₃ |
| A-213 | CH₂CCl₃ |
| A-214 | CF₂CF₃ |
| A-215 | CCl₂CCl₃ |
| A-216 | CHFCH₂CH₃ |
| A-217 | CHClCH₂CH₃ |
| A-218 | CH₂CHFCH₃ |
| A-219 | CH₂CHClCH₃ |
| A-220 | CH₂CH₂CH₂F |
| A-221 | CH₂CH₂CH₂Cl |
| A-222 | CH₂CH₂CH₂Br |
| A-223 | CCl₂CH₂CH₃ |
| A-224 | CF₂CH₂CH₃ |
| A-225 | CH₂CH₂CHF₂ |
| A-226 | CH₂CH₂CHCl₂ |
| A-227 | CH₂CH₂CF₃ |
| A-228 | CH₂CH₂CCl₃ |
| A-229 | CF₂CF₂CF₃ |
| A-230 | CCl₂CCl₂CCl₃ |
| A-231 | CH(CH₃)CF₃ |
| A-232 | CH(CH₃)CH₂F |
| A-233 | CH(CH₃)CH₂Cl |
| A-234 | CH(CH₃)CH₂Br |
| A-235 | CH(CH₃)CHF₂ |
| A-236 | CH(CH₃)CHCl₂ |
| A-237 | CH(CH₂F)₂ |
| A-238 | CH(CH₂Cl)₂ |
| A-239 | CH(CH₂Br)₂ |
| A-240 | CH(CHF₂)₂ |
| A-241 | CH(CHCl₂)₂ |
| A-242 | CHFCH₂CH₂CH₃ |
| A-243 | CHClCH₂CH₂CH₃ |
| A-244 | CH₂CHFCH₂CH₃ |
| A-245 | CH₂CHClCH₂CH₃ |
| A-246 | CH₂CH₂CHFCH₃ |
| A-247 | CH₂CH₂CHClCH₃ |
| A-248 | CH₂CH₂CH₂CH₂F |
| A-249 | CH₂CH₂CH₂CH₂Cl |
| A-250 | CH₂CH₂CH₂CH₂Br |
| A-251 | CCl₂CH₂CH₂CH₃ |
| A-252 | CF₂CH₂CH₂CH₃ |
| A-253 | CH₂CH₂CH₂CHF₂ |
| A-254 | CH₂CH₂CH₂CHCl₂ |
| A-255 | CH₂CH₂CH₂CF₃ |
| A-256 | CH₂CH₂CH₂CCl₃ |
| A-257 | CF₂CF₂CF₂CF₃ |
| A-258 | CCl₂CCl₂CCl₂CCl₃ |
| A-259 | CH(CH₃)CH₂CH₂F |
| A-260 | CH(CH₃)CH₂CH₂Cl |
| A-261 | CH(CH₃)CH₂CH₂Br |
| A-262 | CH(CH₃)CH₂CF₃ |
| A-263 | CHFCH₂CH₂CH₂CH₃ |
| A-264 | CHClCH₂CH₂CH₂CH₃ |
| A-265 | CH₂CHFCH₂CH₂CH₃ |
| A-266 | CH₂CHClCH₂CH₂CH₃ |
| A-267 | CH₂CH₂CHFCH₂CH₃ |
| A-268 | CH₂CH₂CHClCH₂CH₃ |
| A-269 | CH₂CH₂CH₂CHFCH₃ |
| A-270 | CH₂CH₂CH₂CHClCH₃ |
| A-271 | CH₂CH₂CH₂CH₂CH₂F |
| A-272 | CH₂CH₂CH₂CH₂CH₂Cl |
| A-273 | CH₂CH₂CH₂CH₂CH₂Br |
| A-274 | CCl₂CH₂CH₂CH₂CH₃ |
| A-275 | CF₂CH₂CH₂CH₂CH₃ |
| A-276 | CH₂CH₂CH₂CH₂CHF₂ |
| A-277 | CH₂CH₂CH₂CH₂CHCl₂ |
| A-278 | CH₂CH₂CH₂CH₂CF₃ |
| A-279 | CH₂CH₂CH₂CH₂CCl₃ |
| A-280 | CF₂CF₂CF₂CF₂CF₃ |
| A-281 | CCl₂CCl₂CCl₂CCl₂CCl₃ |
| A-282 | CH(CH₃)CH₂CH₂CH₂F |
| A-283 | CH(CH₃)CH₂CH₂CH₂Cl |
| A-284 | CH(CH₃)CH₂CH₂CH₂Br |
| A-285 | CH(CH₃)CH₂CH₂CF₃ |
| A-286 | CHFCH₂CH₂CH₂CH₂CH₃ |
| A-287 | CHClCH₂CH₂CH₂CH₂CH₃ |
| A-288 | CH₂CHFCH₂CH₂CH₂CH₃ |
| A-289 | CH₂CHClCH₂CH₂CH₂CH₃ |
| A-290 | CH₂CH₂CHFCH₂CH₂CH₃ |
| A-291 | CH₂CH₂CHClCH₂CH₂CH₃ |
| A-292 | CH₂CH₂CH₂CHFCH₂CH₃ |
| A-293 | CH₂CH₂CH₂CHClCH₂CH₃ |
| A-294 | CH₂CH₂CH₂CH₂CHFCH₃ |
| A-295 | CH₂CH₂CH₂CH₂CHClCH₃ |
| A-296 | CH₂CH₂CH₂CH₂CH₂CH₂F |
| A-297 | CH₂CH₂CH₂CH₂CH₂CH₂Cl |
| A-298 | CH₂CH₂CH₂CH₂CH₂CH₂Br |
| A-299 | CCl₂CH₂CH₂CH₂CH₂CH₃ |
| A-300 | CF₂CH₂CH₂CH₂CH₂CH₃ |
| A-301 | CH₂CH₂CH₂CH₂CH₂CHF₂ |
| A-302 | CH₂CH₂CH₂CH₂CH₂CHCl₂ |
| A-303 | CH₂CH₂CH₂CH₂CH₂CF₃ |
| A-304 | CH₂CH₂CH₂CH₂CH₂CCl₃ |
| A-305 | CF₂CF₂CF₂CF₂CF₂CF₃ |
| A-306 | CCl₂CCl₂CCl₂CCl₂CCl₂CCl₃ |
| A-307 | CH(CH₃)CH₂CH₂CH₂CH₂F |
| A-308 | CH(CH₃)CH₂CH₂CH₂CH₂Cl |
| A-309 | CH(CH₃)CH₂CH₂CH₂CH₂Br |
| A-310 | CH(CH₃)CH₂CH₂CH₂CF₃ |
| A-311 | CHFCH₂CH₂CH₂CH₂CH₂CH₃ |
| A-312 | CHClCH₂CH₂CH₂CH₂CH₂CH₃ |
| A-313 | CH₂CHFCH₂CH₂CH₂CH₂CH₃ |
| A-314 | CH₂CHClCH₂CH₂CH₂CH₂CH₃ |
| A-315 | CH₂CH₂CH₂CHFCH₂CH₂CH₃ |
| A-316 | CH₂CH₂CH₂CHClCH₂CH₂CH₃ |
| A-317 | CH₂CH₂CH₂CH₂CHFCH₂CH₃ |
| A-318 | CH₂CH₂CH₂CH₂CHClCH₂CH₃ |
| A-319 | CH₂CH₂CH₂CH₂CH₂CHFCH₃ |
| A-320 | CH₂CH₂CH₂CH₂CH₂CHClCH₃ |
| A-321 | CH₂CH₂CH₂CH₂CH₂CH₂CH₂F |
| A-322 | CH₂CH₂CH₂CH₂CH₂CH₂CH₂Cl |
| A-323 | CH₂CH₂CH₂CH₂CH₂CH₂CH₂Br |
| A-324 | CCl₂CH₂CH₂CH₂CH₂CH₂CH₃ |
| A-325 | CF₂CH₂CH₂CH₂CH₂CH₂CH₃ |
| A-326 | CH₂CH₂CH₂CH₂CH₂CH₂CHF₂ |
| A-327 | CH₂CH₂CH₂CH₂CH₂CH₂CHCl₂ |
| A-328 | CH₂CH₂CH₂CH₂CH₂CH₂CF₃ |
| A-329 | CH₂CH₂CH₂CH₂CH₂CH₂CCl₃ |
| A-330 | CF₂CF₂CF₂CF₂CF₂CF₂CF₃ |
| A-331 | CCl₂CCl₂CCl₂CCl₂CCl₂CCl₂CCl₃ |
| A-332 | CH(CH₃)CH₂CH₂CH₂CH₂CH₂F |
| A-333 | CH(CH₃)CH₂CH₂CH₂CH₂CH₂Cl |
| A-334 | CH(CH₃)CH₂CH₂CH₂CH₂CH₂Br |
| A-335 | CH(CH₃)CH₂CH₂CH₂CH₂CF₃ |
| A-336 | CHFCH₂CH₂CH₂CH₂CH₂CH₂CH₃ |
| A-337 | CHClCH₂CH₂CH₂CH₂CH₂CH₂CH₃ |
| A-338 | CH₂CHFCH₂CH₂CH₂CH₂CH₂CH₃ |
| A-339 | CH₂CHClCH₂CH₂CH₂CH₂CH₂CH₃ |
| A-340 | CH₂CH₂CHFCH₂CH₂CH₂CH₂CH₃ |
| A-341 | CH₂CH₂CHClCH₂CH₂CH₂CH₂CH₃ |
| A-342 | CH₂CH₂CH₂CH₂CHFCH₂CH₂CH₃ |
| A-343 | CH₂CH₂CH₂CH₂CHClCH₂CH₂CH₃ |
| A-344 | CH₂CH₂CH₂CH₂CH₂CHFCH₂CH₃ |
| A-345 | CH₂CH₂CH₂CH₂CH₂CHClCH₂CH₃ |
| A-346 | CH₂CH₂CH₂CH₂CH₂CH₂CHFCH₃ |
| A-347 | CH₂CH₂CH₂CH₂CH₂CH₂CHClCH₃ |
| A-348 | CH₂CH₂CH₂CH₂CH₂CH₂CH₂CH₂F |
| A-349 | CH₂CH₂CH₂CH₂CH₂CH₂CH₂CH₂Cl |
| A-350 | CH₂CH₂CH₂CH₂CH₂CH₂CH₂CH₂Br |
| A-351 | CCl₂CH₂CH₂CH₂CH₂CH₂CH₂CH₃ |
| A-352 | CF₂CH₂CH₂CH₂CH₂CH₂CH₂CH₃ |
| A-353 | CH₂CH₂CH₂CH₂CH₂CH₂CH₂CHF₂ |
| A-354 | CH₂CH₂CH₂CH₂CH₂CH₂CH₂CHCl₂ |
| A-355 | CH₂CH₂CH₂CH₂CH₂CH₂CH₂CF₃ |
| A-356 | CH₂CH₂CH₂CH₂CH₂CH₂CH₂CCl₃ |
| A-357 | CF₂CF₂CF₂CF₂CF₂CF₂CF₂CF₃ |
| A-358 | CCl₂CCl₂CCl₂CCl₂CCl₂CCl₂CCl₂CCl₃ |
| A-359 | CH(CH₃)CH₂CH₂CH₂CH₂CH₂CH₂F |
| A-360 | CH(CH₃)CH₂CH₂CH₂CH₂CH₂CH₂Cl |
| A-361 | CH(CH₃)CH₂CH₂CH₂CH₂CH₂CH₂Br |
| A-362 | CH(CH₃)CH₂CH₂CH₂CH₂CH₂CF₃ |
| A-363 | CHFCH₂CH₂CH₂CH₂CH₂CH₂CH₂CH₃ |
| A-364 | CHClCH₂CH₂CH₂CH₂CH₂CH₂CH₂CH₃ |
| A-365 | CH₂CHFCH₂CH₂CH₂CH₂CH₂CH₂CH₃ |
| A-366 | CH₂CHClCH₂CH₂CH₂CH₂CH₂CH₂CH₃ |
| A-367 | CH₂CH₂CHFCH₂CH₂CH₂CH₂CH₂CH₃ |
| A-368 | CH₂CH₂CHClCH₂CH₂CH₂CH₂CH₂CH₃ |
| A-369 | CH₂CH₂CH₂CHFCH₂CH₂CH₂CH₂CH₃ |
| A-370 | CH₂CH₂CH₂CHClCH₂CH₂CH₂CH₂CH₃ |
| A-371 | CH₂CH₂CH₂CH₂CH₂CHFCH₂CH₂CH₃ |
| A-372 | CH₂CH₂CH₂CH₂CH₂CHClCH₂CH₂CH₃ |
| A-373 | CH₂CH₂CH₂CH₂CH₂CH₂CHFCH₂CH₃ |
| A-374 | CH₂CH₂CH₂CH₂CH₂CH₂CHClCH₂CH₃ |
| A-375 | CH₂CH₂CH₂CH₂CH₂CH₂CH₂CHFCH₃ |
| A-376 | CH₂CH₂CH₂CH₂CH₂CH₂CH₂CHClCH₃ |
| A-377 | CH₂CH₂CH₂CH₂CH₂CH₂CH₂CH₂CH₂F |
| A-378 | CH₂CH₂CH₂CH₂CH₂CH₂CH₂CH₂CH₂Cl |
| A-379 | CH₂CH₂CH₂CH₂CH₂CH₂CH₂CH₂CH₂Br |
| A-380 | CCl₂CH₂CH₂CH₂CH₂CH₂CH₂CH₂CH₃ |
| A-381 | CF₂CH₂CH₂CH₂CH₂CH₂CH₂CH₂CH₃ |
| A-382 | CH₂CH₂CH₂CH₂CH₂CH₂CH₂CH₂CHF₂ |
| A-383 | CH₂CH₂CH₂CH₂CH₂CH₂CH₂CH₂CHCl₂ |
| A-384 | CH₂CH₂CH₂CH₂CH₂CH₂CH₂CH₂CF₃ |
| A-385 | CH₂CH₂CH₂CH₂CH₂CH₂CH₂CH₂CCl₃ |
| A-386 | CF₂CF₂CF₂CF₂CF₂CF₂CF₂CF₂CF₃ |
| A-387 | CCl₂CCl₂CCl₂CCl₂CCl₂CCl₂CCl₂CCl₂CCl₃ |
| A-388 | CH(CH₃)CH₂CH₂CH₂CH₂CH₂CH₂CH₂F |
| A-389 | CH(CH₃)CH₂CH₂CH₂CH₂CH₂CH₂CH₂Cl |
| A-390 | CH(CH₃)CH₂CH₂CH₂CH₂CH₂CH₂CH₂Br |
| A-391 | CH(CH₃)CH₂CH₂CH₂CH₂CH₂CH₂CF₃ |
| A-392 | CHFCH₂CH₂CH₂CH₂CH₂CH₂CH₂CH₂CH₃ |
| A-393 | CHClCH₂CH₂CH₂CH₂CH₂CH₂CH₂CH₂CH₃ |
| A-394 | CH₂CHFCH₂CH₂CH₂CH₂CH₂CH₂CH₂CH₃ |
| A-395 | CH₂CHClCH₂CH₂CH₂CH₂CH₂CH₂CH₂CH₃ |
| A-396 | CH₂CH₂CHFCH₂CH₂CH₂CH₂CH₂CH₂CH₃ |
| A-397 | CH₂CH₂CHClCH₂CH₂CH₂CH₂CH₂CH₂CH₃ |
| A-398 | CH₂CH₂CH₂CHFCH₂CH₂CH₂CH₂CH₂CH₃ |
| A-399 | CH₂CH₂CH₂CHClCH₂CH₂CH₂CH₂CH₂CH₃ |
| A-400 | CH₂CH₂CH₂CH₂CHFCH₂CH₂CH₂CH₂CH₃ |
| A-401 | CH₂CH₂CH₂CH₂CHClCH₂CH₂CH₂CH₂CH₃ |
| A-402 | CH₂CH₂CH₂CH₂CH₂CH₂CHFCH₂CH₂CH₃ |
| A-403 | CH₂CH₂CH₂CH₂CH₂CH₂CHClCH₂CH₂CH₃ |
| A-404 | CH₂CH₂CH₂CH₂CH₂CH₂CH₂CHFCH₂CH₃ |
| A-405 | CH₂CH₂CH₂CH₂CH₂CH₂CH₂CHClCH₂CH₃ |
| A-406 | CH₂CH₂CH₂CH₂CH₂CH₂CH₂CH₂CHFCH₃ |
| A-407 | CH₂CH₂CH₂CH₂CH₂CH₂CH₂CH₂CHClCH₃ |
| A-408 | CH₂CH₂CH₂CH₂CH₂CH₂CH₂CH₂CH₂CH₂F |
| A-409 | CH₂CH₂CH₂CH₂CH₂CH₂CH₂CH₂CH₂CH₂Br |
| A-410 | CH₂CH₂CH₂CH₂CH₂CH₂CH₂CH₂CH₂CH₂Cl |
| A-411 | CCl₂CH₂CH₂CH₂CH₂CH₂CH₂CH₂CH₂CH₃ |
| A-412 | CF₂CH₂CH₂CH₂CH₂CH₂CH₂CH₂CH₂CH₃ |
| A-413 | CH₂CH₂CH₂CH₂CH₂CH₂CH₂CH₂CH₂CHF₂ |
| A-414 | CH₂CH₂CH₂CH₂CH₂CH₂CH₂CH₂CH₂CHCl₂ |
| A-415 | CH₂CH₂CH₂CH₂CH₂CH₂CH₂CH₂CH₂CF₃ |
| A-416 | CH₂CH₂CH₂CH₂CH₂CH₂CH₂CH₂CH₂CCl₃ |
| A-417 | CF₂CF₂CF₂CF₂CF₂CF₂CF₂CF₂CF₂CF₃ |
| A-418 | CCl₂CCl₂CCl₂CCl₂CCl₂CCl₂CCl₂CCl₂CCl₂CCl₃ |
| A-419 | CH(CH₃)CH₂CH₂CH₂CH₂CH₂CH₂CH₂CH₂F |
| A-420 | CH(CH₃)CH₂CH₂CH₂CH₂CH₂CH₂CH₂CH₂Cl |
| A-421 | CH(CH₃)CH₂CH₂CH₂CH₂CH₂CH₂CH₂CH₂Br |
| A-422 | CH(CH₃)CH₂CH₂CH₂CH₂CH₂CH₂CH₂CF₃ |
| A-423 | CH═CH₂ |
| A-424 | CH₂CH═CH₂ |
| A-425 | CH═CHCH₃ |
| A-426 | C(CH₃)═CH₂ |
| A-427 | CH₂CH₂CH═CH₂ |
| A-428 | CH₂CH═CHCH₃ |
| A-429 | CH═CHCH₂CH₃ |
| A-430 | CH(CH₃)CH═CH₂ |
| A-431 | C(CH₃)═CHCH₃ |
| A-432 | CH═C(CH₃)₂ |
| A-433 | CH₂CH₂CH₂CH═CH₂ |
| A-434 | CH₂CH₂CH═CHCH₃ |
| A-435 | CH₂CH═CHCH₂CH₃ |
| A-436 | CH═CHCH₂CH₂CH₃ |
| A-437 | CH(CH₃)CH₂CH═CH₂ |
| A-438 | CH₂C(CH₃)═CHCH₃ |
| A-439 | CH₂CH═C(CH₃)₂ |
| A-440 | CH₂CH₂CH₂CH₂CH═CH₂ |
| A-441 | CH₂CH₂CH₂CH═CHCH₃ |
| A-442 | CH₂CH₂CH═CHCH₂CH₃ |
| A-443 | CH₂CH═CHCH₂CH₂CH₃ |
| A-444 | CH═CHCH₂CH₂CH₂CH₃ |
| A-445 | CH(CH₃)CH₂CH₂CH═CH₂ |
| A-446 | CH(CH₃)CH₂CH═CHCH₃ |
| A-447 | CH₂C(CH₃)═CHCH₂CH₃ |
| A-448 | CH₂CH₂CH═C(CH₃)₂ |
| A-449 | CH₂CH₂CH₂CH₂CH₂CH═CH₂ |
| A-450 | CH₂CH₂CH₂CH₂CH═CHCH₃ |
| A-451 | CH₂CH₂CH₂CH═CHCH₂CH₃ |
| A-452 | CH₂CH₂CH═CHCH₂CH₂CH₃ |
| A-453 | CH₂CH═CHCH₂CH₂CH₂CH₃ |
| A-454 | CH═CHCH₂CH₂CH₂CH₂CH₃ |
| A-455 | CH(CH₃)CH₂CH₂CH₂CH═CH₂ |
| A-456 | CH(CH₃)CH₂CH₂CH═CHCH₃ |
| A-457 | C(CH₃)═CHCH₂CH₂CH₂CH₃ |
| A-458 | CH₂CH₂CH₂CH═C(CH₃)₂ |
| A-459 | CH₂CH₂CH₂CH₂CH₂CH₂CH═CH₂ |
| A-460 | CH₂CH₂CH₂CH₂CH₂CH═CHCH₃ |
| A-461 | CH₂CH₂CH₂CH₂CH═CHCH₂CH₃ |
| A-462 | CH₂CH₂CH₂CH═CHCH₂CH₂CH₃ |
| A-463 | CH₂CH₂CH═CHCH₂CH₂CH₂CH₃ |
| A-464 | CH₂CH═CHCH₂CH₂CH₂CH₂CH₃ |
| A-465 | CH═CHCH₂CH₂CH₂CH₂CH₂CH₃ |
| A-466 | CH(CH₃)CH₂CH₂CH₂CH₂CH═CH₂ |
| A-467 | CH(CH₃)CH₂CH₂CH₂CH═CHCH₃ |
| A-468 | C(CH₃)═CHCH₂CH₂CH₂CH₂CH₃ |
| A-469 | CH₂CH₂CH₂CH₂CH═C(CH₃)₂ |
| A-470 | CH₂CH₂CH₂CH₂CH₂CH₂CH₂CH═CH₂ |
| A-471 | CH₂CH₂CH₂CH₂CH₂CH₂CH═CHCH₃ |
| A-472 | CH₂CH₂CH₂CH₂CH₂CH═CHCH₂CH₃ |
| A-473 | CH₂CH₂CH₂CH₂CH═CHCH₂CH₂CH₃ |
| A-474 | CH₂CH₂CH₂CH═CHCH₂CH₂CH₂CH₃ |
| A-475 | CH₂CH₂CH═CHCH₂CH₂CH₂CH₂CH₃ |
| A-476 | CH₂CH═CHCH₂CH₂CH₂CH₂CH₂CH₃ |
| A-477 | CH═CHCH₂CH₂CH₂CH₂CH₂CH₂CH₃ |
| A-478 | CH(CH₃)CH₂CH₂CH₂CH₂CH₂CH═CH₂ |
| A-479 | CH(CH₃)CH₂CH₂CH₂CH₂CH═CHCH₃ |
| A-480 | C(CH₃)═CHCH₂CH₂CH₂CH₂CH₂CH₃ |
| A-481 | CH₂CH₂CH₂CH₂CH₂CH═C(CH₃)₂ |
| A-482 | CH₂CH₂CH₂CH₂CH₂CH₂CH₂CH₂CH═CH₂ |
| A-483 | CH₂CH₂CH₂CH₂CH₂CH₂CH₂CH═CHCH₃ |
| A-484 | CH₂CH₂CH₂CH₂CH₂CH₂CH═CHCH₂CH₃ |
| A-485 | CH₂CH₂CH₂CH₂CH₂CH═CHCH₂CH₂CH₃ |
| A-486 | CH₂CH₂CH₂CH₂CH═CHCH₂CH₂CH₂CH₃ |
| A-487 | CH₂CH₂CH₂CH═CHCH₂CH₂CH₂CH₂CH₃ |
| A-488 | CH₂CH₂CH═CHCH₂CH₂CH₂CH₂CH₂CH₃ |
| A-489 | CH₂CH═CHCH₂CH₂CH₂CH₂CH₂CH₂CH₃ |
| A-490 | CH═CHCH₂CH₂CH₂CH₂CH₂CH₂CH₂CH₃ |
| A-491 | CH(CH₃)CH₂CH₂CH₂CH₂CH₂CH₂CH═CH₂ |
| A-492 | CH(CH₃)CH₂CH₂CH₂CH₂CH₂CH═CHCH₃ |
| A-493 | C(CH₃)═CHCH₂CH₂CH₂CH₂CH₂CH₂CH₃ |
| A-494 | CH₂CH₂CH₂CH₂CH₂CH₂CH═C(CH₃)₂ |
| A-495 | C≡CH |
| A-496 | CH₂C≡CH |
| A-497 | C≡CCH₃ |
| A-498 | CH₂CH₂C≡CH |
| A-499 | CH₂C≡CCH₃ |
| A-500 | C≡CCH₂CH₃ |
| A-501 | CH(CH₃)C≡CH |
| A-502 | CH₂CH₂CH₂C≡CH |
| A-503 | CH₂CH₂C≡CCH₃ |
| A-504 | CH₂C≡CCH₂CH₃ |
| A-505 | C≡CCH₂CH₂CH₃ |
| A-506 | CH(CH₃)CH₂C≡CH |
| A-507 | CH₂CH₂CH₂CH₂C≡CH |
| A-508 | CH₂CH₂CH₂C≡CCH₃ |
| A-509 | CH₂CH₂C≡CCH₂CH₃ |
| A-510 | CH₂C≡CCH₂CH₂CH₃ |
| A-511 | C≡CCH₂CH₂CH₂CH₃ |
| A-512 | CH(CH₃)CH₂CH₂C≡CH |
| A-513 | CH(CH₃)CH₂C≡CCH₃ |
| A-514 | CH₂CH₂CH₂CH₂CH₂C≡CH |
| A-515 | CH₂CH₂CH₂CH₂C≡CCH₃ |
| A-516 | CH₂CH₂CH₂C≡CCH₂CH₃ |
| A-517 | CH₂CH₂C≡CCH₂CH₂CH₃ |
| A-518 | CH₂C≡CCH₂CH₂CH₂CH₃ |
| A-519 | C≡CCH₂CH₂CH₂CH₂CH₃ |
| A-520 | CH(CH₃)CH₂CH₂CH₂C≡CH |
| A-521 | CH(CH₃)CH₂CH₂C≡CCH₃ |
| A-522 | CH(CH₃)CH₂C≡CCH₂CH₃ |
| A-523 | CH₂CH₂CH₂CH₂CH₂CH₂C≡CH |
| A-524 | CH₂CH₂CH₂CH₂CH₂C≡CCH₃ |
| A-525 | CH₂CH₂CH₂CH₂C≡CCH₂CH₃ |
| A-526 | CH₂CH₂CH₂C≡CCH₂CH₂CH₃ |
| A-527 | CH₂CH₂C≡CCH₂CH₂CH₂CH₃ |
| A-528 | CH₂C≡CCH₂CH₂CH₂CH₂CH₃ |
| A-529 | C≡CCH₂CH₂CH₂CH₂CH₂CH₃ |
| A-530 | CH(CH₃)CH₂CH₂CH₂CH₂C≡CH |
| A-531 | CH(CH₃)CH₂CH₂CH₂C≡CCH₃ |
| A-532 | CH₂CH₂CH₂CH₂CH₂CH₂CH₂C≡CH |
| A-533 | CH₂CH₂CH₂CH₂CH₂CH₂C≡CCH₃ |
| A-534 | CH₂CH₂CH₂CH₂CH₂C≡CCH₂CH₃ |
| A-535 | CH₂CH₂CH₂CH₂C≡CCH₂CH₂CH₃ |
| A-536 | CH₂CH₂CH₂C≡CCH₂CH₂CH₂CH₃ |
| A-537 | CH₂CH₂C≡CCH₂CH₂CH₂CH₂CH₃ |
| A-538 | CH₂C≡CCH₂CH₂CH₂CH₂CH₂CH₃ |
| A-539 | C≡CCH₂CH₂CH₂CH₂CH₂CH₂CH₃ |
| A-540 | CH(CH₃)CH₂CH₂CH₂CH₂CH₂C≡CH |
| A-541 | CH(CH₃)CH₂CH₂CH₂CH₂C≡CCH₃ |
| A-542 | CH₂CH₂CH₂CH₂CH₂CH₂CH₂CH₂C≡CH |
| A-543 | CH₂CH₂CH₂CH₂CH₂CH₂CH₂C≡CCH₃ |
| A-544 | CH₂CH₂CH₂CH₂CH₂CH₂C≡CCH₂CH₃ |
| A-545 | CH₂CH₂CH₂CH₂CH₂C≡CCH₂CH₂CH₃ |
| A-546 | CH₂CH₂CH₂CH₂C≡CCH₂CH₂CH₂CH₃ |
| A-547 | CH₂CH₂CH₂C≡CCH₂CH₂CH₂CH₂CH₃ |
| A-548 | CH₂CH₂C≡CCH₂CH₂CH₂CH₂CH₂CH₃ |
| A-549 | CH₂C≡CCH₂CH₂CH₂CH₂CH₂CH₂CH₃ |
| A-550 | C≡CCH₂CH₂CH₂CH₂CH₂CH₂CH₂CH₃ |
| A-551 | CH(CH₃)CH₂CH₂CH₂CH₂CH₂CH₂C≡CH |
| A-552 | CH(CH₃)CH₂CH₂CH₂CH₂CH₂C≡CCH₃ |
| A-553 | CH₂CH₂CH₂CH₂CH₂CN |
| A-554 | CH(CH₃)CH₂CH₂CH₂CN |
| A-555 | CH₂CH(CH₃)CH₂CH₂CN |
| A-556 | CH₂CH₂CH(CH₃)CH₂CN |
| A-557 | CH₂CH₂CH(CH₃)CH₂CN |
| A-558 | CH(CH₃)CH(CH₃)CH₂CN |
| A-559 | CH(CH₃)CH(CH₃)CH₂CN |
| A-560 | CH₂C(CH₃)₂CH₂CN |
| A-561 | CH₂CH₂CH₂CH₂CH₂CH₂CN |
| A-562 | CH(CH₃)CH₂CH₂CH₂CH₂CN |
| A-563 | CH₂CH(CH₃)CH₂CH₂CH₂CN |
| A-564 | CH₂CH₂CH(CH₃)CH₂CH₂CN |
| A-565 | CH₂CH₂CH(CH₃)₂CH₂CH₂CN |
| A-566 | CH₂CH₂CH₂CH(CH₃)CH₂CN |
| A-567 | CH(CH₃)CH(CH₃)CH₂CH₂CN |
| A-568 | CH(CH₃)CH₂CH(CH₃)CH₂CN |
| A-569 | CH₂CH₂C(CH₃)₂CH₂CN |
| A-570 | CH(CH₃)CH₂CH(CH₃)CH₂CN |
| A-571 | CH₂CH₂CH₂CH₂CH₂CH₂CH₂CN |
| A-572 | CH(CH₃)CH₂CH₂CH₂CH₂CH₂CN |
| A-573 | CH₂CH(CH₃)CH₂CH₂CH₂CH₂CN |
| A-574 | CH₂CH₂CH(CH₃)CH₂CH₂CH₂CN |
| A-575 | CH₂CH₂CH₂CH(CH₃)CH₂CH₂CN |
| A-576 | CH₂CH₂CH₂CH₂CH(CH₃)CH₂CN |
| A-577 | CH(CH₃)CH(CH₃)CH₂CH₂CH₂CN |
| A-578 | CH₂CH(CH₃)CH(CH₃)CH₂CH₂CN |
| A-579 | CH₂CH₂CH₂C(CH₃)₂CH₂CN |
| A-580 | CH(CH₃)CH₂CH(CH₃)CH₂CH₂CN |
| A-581 | CH₂CH(CH₃)CH(CH₃)CH₂CH₂CN |
| A-582 | CH(CH₃)CH₂CH₂CH(CH₃)CH₂CN |
| A-583 | CH₂CH₂CH₂CH₂CH₂CH₂CH₂CH₂CN |
| A-584 | CH(CH₃)CH₂CH₂CH₂CH₂CH₂CH₂CN |
| A-585 | CH₂CH(CH₃)CH₂CH₂CH₂CH₂CH₂CN |
| A-586 | CH₂CH₂CH(CH₃)CH₂CH₂CH₂CH₂CN |
| A-587 | CH₂CH₂CH₂CH(CH₃)CH₂CH₂CH₂CN |
| A-588 | CH₂CH₂CH₂CH₂CH(CH₃)CH₂CH₂CN |
| A-589 | CH₂CH₂CH₂CH₂CH₂CH(CH₃)CH₂CN |
| A-590 | CH₂CH₂CH₂CH₂C(CH₃)₂CH₂CN |
| A-591 | CH(CH₃)CH(CH₃)CH₂CH₂CH₂CH₂CN |
| A-592 | CH₂CH(CH₃)CH(CH₃)CH₂CH₂CH₂CN |
| A-593 | CH₂CH₂CH₂C(CH₃)₂CH₂CH₂CN |
| A-594 | CH(CH₃)CH₂CH(CH₃)CH₂CH₂CH₂CN |
| A-595 | CH₂CH(CH₃)CH(CH₃)CH₂CH₂CH₂CN |
| A-596 | CH(CH₃)CH₂CH₂CH(CH₃)CH₂CH₂CN |
| A-597 | CH(CH₃)CH₂CH₂CH₂CH(CH₃)CH₂CN |
| A-598 | CH₂CH₂CH₂CH₂CH₂CH₂CH₂CH₂CH₂CN |
| A-599 | CH(CH₃)CH₂CH₂CH₂CH₂CH₂CH₂CH₂CN |
| A-600 | CH₂CH(CH₃)CH₂CH₂CH₂CH₂CH₂CH₂CN |
| A-601 | CH₂CH₂CH(CH₃)CH₂CH₂CH₂CH₂CH₂CN |
| A-602 | CH₂CH₂CH₂CH(CH₃)CH₂CH₂CH₂CH₂CN |
| A-603 | CH₂CH₂CH₂CH₂CH(CH₃)CH₂CH₂CH₂CN |
| A-604 | CH₂CH₂CH₂CH₂CH₂CH₂C(CH₃)₂CH₂CN |
| A-605 | CH(CH₃)CH(CH₃)CH₂CH₂CH₂CH₂CH₂CN |
| A-606 | CH₂CH(CH₃)CH(CH₃)CH₂CH₂CH₂CH₂CN |
| A-607 | CH₂CH₂CH₂C(CH₃)₂CH₂CH₂CH₂CN |
| A-608 | CH(CH₃)CH₂CH(CH₃)CH₂CH₂CH₂CH₂CN |
| A-609 | CH₂CH(CH₃)CH(CH₃)CH₂CH₂CH₂CH₂CN |
| A-610 | CH(CH₃)CH₂CH₂CH(CH₃)CH₂CH₂CH₂CN |
| A-611 | CH(CH₃)CH₂CH₂CH₂C(CH₃)₂CH₂CN |
| A-612 | CH₂CH(CH₃)CH₂CH₂CH(CH₃)₂CH₂CN |
| A-613 | CH(CH₃)CH₂CH₂CH₂CH₂CH(CH₃)CH₂CN |
| A-614 | CH₂CH(CH₃)CH₂CH₂CH₂CH(CH₃)CH₂CN |
| A-615 | CH₂CH₂CH₂CH₂CH₂CH₂CH₂CH₂CH₂CH₂CN |
| A-616 | CH(CH₃)CH₂CH₂CH₂CH₂CH₂CH₂CH₂CH₂CN |
| A-617 | CH₂CH(CH₃)CH₂CH₂CH₂CH₂CH₂CH₂CH₂CN |
| A-618 | CH₂CH₂CH(CH₃)CH₂CH₂CH₂CH₂CH₂CH₂CN |
| A-619 | CH₂CH₂CH(CH₃)CH₂CH₂CH₂CH₂CH₂CN |
| A-620 | CH₂CH₂CH₂CH(CH₃)CH₂CH₂CH₂CH₂CN |
| A-621 | CH₂CH₂CH₂CH₂CH₂C(CH₃)₂CH₂CN |
| A-622 | CH(CH₃)CH(CH₃)CH₂CH₂CH₂CH₂CH₂CH₂CN |
| A-623 | CH₂CH(CH₃)CH(CH₃)CH₂CH₂CH₂CH₂CH₂CN |
| A-624 | CH₂CH₂CH₂C(CH₃)₂CH₂CH₂CH₂CH₂CN |
| A-625 | CH(CH₃)CH₂CH(CH₃)CH₂CH₂CH₂CH₂CH₂CN |
| A-626 | CH₂CH(CH₃)CH(CH₃)CH₂CH₂CH₂CH₂CH₂CN |
| A-627 | CH(CH₃)CH₂CH₂CH(CH₃)CH₂CH₂CH₂CH₂CN |
| A-628 | CH(CH₃)CH₂CH₂CH₂CH(CH₃)CH₂CH₂CH₂CN |
| A-629 | CH(CH₃)CH₂CH₂CH₂CH₂CH(CH₃)CH₂CH₂CN |
| A-630 | CH(CH₃)CH₂CH₂CH₂CH₂CH₂CH(CH₃)CH₂CN |
| A-631 | CH(CH₃)CH₂CH₂CH₂CH₂CH₂C(CH₃)CH₂CN |
| A-632 | CH₂CH(CH₃)CH₂CH₂CH₂CH₂CH(CH₃)CH₂CN |
| A-633 | CH(CH₃)CH₂CH₂CH₂CH₂C(CH₃)₂CH₂CN |
| A-634 | CH₂CH(CH₃)CH₂CH₂CH₂C(CH₃)₂CH₂CN |
| A-635 | CH₂CH₂CH₂CH₂CH₂CH₂CH₂CH₂CH₂CH₂CH₂CN |
| A-636 | CH(CH₃)CH₂CH₂CH₂CH₂CH₂CH₂CH₂CH₂CH₂CN |
| A-637 | CH₂CH(CH₃)CH₂CH₂CH₂CH₂CH₂CH₂CH₂CH₂CN |
| A-638 | CH₂CH₂CH(CH₃)CH₂CH₂CH₂CH₂CH₂CH₂CH₂CN |
| A-639 | CH₂CH₂CH₂CH(CH₃)CH₂CH₂CH₂CH₂CH₂CH₂CN |
| A-640 | CH₂CH₂CH₂CH₂CH(CH₃)CH₂CH₂CH₂CH₂CH₂CN |
| A-641 | CH₂CH₂CH₂CH₂CH₂CH₂CH₂C(CH₃)₂CH₂CN |
| A-642 | CH(CH₃)CH(CH₃)CH₂CH₂CH₂CH₂CH₂CH₂CH₂CN |
| A-643 | CH₂CH(CH₃)CH(CH₃)CH₂CH₂CH₂CH₂CH₂CH₂CN |
| A-644 | CH₂CH₂CH₂C(CH₃)₂CH₂CH₂CH₂CH₂CH₂CN |
| A-645 | CH(CH₃)CH₂CH(CH₃)CH₂CH₂CH₂CH₂CH₂CH₂CN |
| A-646 | CH₂CH(CH₃)CH(CH₃)CH₂CH₂CH₂CH₂CH₂CH₂CN |
| A-647 | CH(CH₃)CH₂CH₂CH(CH₃)CH₂CH₂CH₂CH₂CH₂CN |
| A-648 | CH(CH₃)CH₂CH₂CH₂CH(CH₃)CH₂CH₂CH₂CH₂CN |
| A-649 | CH(CH₃)CH₂CH₂CH₂CH₂CH(CH₃)CH₂CH₂CH₂CN |
| A-650 | CH(CH₃)CH₂CH₂CH₂CH₂CH₂CH(CH₃)CH₂CH₂CN |
| A-651 | CH(CH₃)CH₂CH₂CH₂CH₂CH₂CH₂CH(CH₃)CH₂CN |
| A-652 | CH₂CH(CH₃)CH₂CH₂CH₂CH₂CH(CH₃)CH₂CH₂CN |
| A-653 | CH₂CH(CH₃)CH₂CH₂CH₂CH₂CH(CH₃)CH₂CH₂CN |
| A-654 | CH₂CH₂CH(CH₃)CH₂CH₂CH₂CH₂CH(CH₃)CH₂CN |
| A-655 | CH₂CH(CH₃)CH₂CH₂CH₂CH₂C(CH₃)₂CH₂CN |
| A-656 | CH₂CH₂CH₂CH₂CH₂CH₂CH₂CH₂CH₂CH₂CH₂CH₂CN |
| A-657 | CH(CH₃)CH₂CH₂CH₂CH₂CH₂CH₂CH₂CH₂CH₂CH₂CN |
| A-658 | CH₂CH(CH₃)CH₂CH₂CH₂CH₂CH₂CH₂CH₂CH₂CH₂CN |
| A-659 | CH₂CH₂CH(CH₃)CH₂CH₂CH₂CH₂CH₂CH₂CH₂CH₂CN |
| A-660 | CH₂CH₂CH₂CH(CH₃)CH₂CH₂CH₂CH₂CH₂CH₂CH₂CN |
| A-661 | CH₂CH₂CH₂CH₂CH(CH₃)CH₂CH₂CH₂CH₂CH₂CH₂CN |
| A-662 | CH₂CH₂CH₂CH₂CH₂CH(CH₃)CH₂CH₂CH₂CH₂CH₂CN |
| A-663 | CH₂CH₂CH₂CH₂CH₂CH₂CH(CH₃)CH₂CH₂CH₂CH₂CN |
| A-664 | CH₂CH₂CH₂CH₂CH₂CH₂CH₂CH(CH₃)CH₂CH₂CH₂CN |
| A-665 | CH₂CH₂CH₂CH₂CH₂CH₂CH₂CH₂CH(CH₃)CH₂CH₂CN |
| A-666 | CH₂CH₂CH₂CH₂CH₂CH₂CH₂CH₂CH₂CH(CH₃)CH₂CN |
| A-667 | CH(CH₃)CH(CH₃)CH₂CH₂CH₂CH₂CH₂CH₂CH₂CH₂CN |
| A-668 | CH₂CH(CH₃)CH(CH₃)CH₂CH₂CH₂CH₂CH₂CH₂CH₂CN |
| A-669 | CH₂CH₂CH₂C(CH₃)₂CH₂CH₂CH₂CH₂CH₂CH₂CN |
| A-670 | CH₂CH₂CH₂CH₂CH(CH₃)CH₂CH₂CH₂CH₂CH₂CH₂CN |
| A-671 | CH(CH₃)CH₂CH(CH₃)CH₂CH₂CH₂CH₂CH₂CH₂CH₂CN |
| A-672 | CH(CH₃)CH₂CH₂CH(CH₃)CH₂CH₂CH₂CH₂CH₂CH₂CN |
| A-673 | CH(CH₃)CH₂CH₂CH₂CH(CH₃)CH₂CH₂CH₂CH₂CH₂CN |
| A-674 | CH(CH₃)CH₂CH₂CH₂CH₂CH(CH₃)CH₂CH₂CH₂CH₂CN |
| A-675 | CH(CH₃)CH₂CH₂CH₂CH₂CH₂CH(CH₃)CH₂CH₂CH₂CN |
| A-676 | CH(CH₃)CH₂CH₂CH₂CH₂CH₂CH₂CH(CH₃)CH₂CH₂CN |
| A-677 | CH₂CH(CH₃)CH₂CH₂CH₂CH₂CH(CH₃)CH₂CH₂CH₂CN |
| A-678 | CH₂CH₂CH(CH₃)CH₂CH₂CH₂CH₂CH(CH₃)CH₂CH₂CN |
| A-679 | CH₂CH₂CH₂CH(CH₃)CH₂CH₂CH₂CH(CH₃)CH₂CH₂CN |
| A-680 | CH₂CH(CH₃)CH₂CH₂CH₂CH₂CH₂C(CH₃)₂CH₂CN |
| A-681 | CHFCH₂CN |
| A-682 | CHClCH₂CN |
| A-683 | CCl₂CH₂CN |
| A-684 | CF₂CH₂CN |
| A-685 | CHFCH₂CH₂CN |
| A-686 | CHClCH₂CH₂CN |
| A-687 | CCl₂CH₂CH₂CN |
| A-688 | CF₂CH₂CH₂CN |
| A-689 | CHFCH₂CH₂CH₂CN |
| A-690 | CHClCH₂CH₂CH₂CN |
| A-691 | CCl₂CH₂CH₂CH₂CN |
| A-692 | CF₂CH₂CH₂CH₂CN |
| A-693 | CHFCH₂CH₂CH₂CH₂CN |
| A-694 | CHClCH₂CH₂CH₂CH₂CN |
| A-695 | CCl₂CH₂CH₂CH₂CH₂CN |
| A-696 | CF₂CH₂CH₂CH₂CH₂CN |
| A-697 | CHFCH₂CH₂CH₂CH₂CH₂CN |
| A-698 | CHClCH₂CH₂CH₂CH₂CH₂CN |
| A-699 | CCl₂CH₂CH₂CH₂CH₂CH₂CN |
| A-700 | CF₂CH₂CH₂CH₂CH₂CH₂CN |
| A-701 | CHFCH₂CH₂CH₂CH₂CH₂CH₂CN |
| A-702 | CHClCH₂CH₂CH₂CH₂CH₂CH₂CN |
| A-703 | CCl₂CH₂CH₂CH₂CH₂CH₂CH₂CN |
| A-704 | CF₂CH₂CH₂CH₂CH₂CH₂CH₂CN |
| A-705 | CHFCH₂CH₂CH₂CH₂CH₂CH₂CH₂CN |
| A-706 | CHClCH₂CH₂CH₂CH₂CH₂CH₂CH₂CN |
| A-707 | CCl₂CH₂CH₂CH₂CH₂CH₂CH₂CH₂CN |
| A-708 | CF₂CH₂CH₂CH₂CH₂CH₂CH₂CH₂CN |
| A-709 | CHFCH₂CH₂CH₂CH₂CH₂CH₂CH₂CH₂CN |
| A-710 | CHClCH₂CH₂CH₂CH₂CH₂CH₂CH₂CH₂CN |
| A-711 | CCl₂CH₂CH₂CH₂CH₂CH₂CH₂CH₂CH₂CN |
| A-712 | CF₂CH₂CH₂CH₂CH₂CH₂CH₂CH₂CH₂CN |
| A-713 | CHFCH₂CH₂CH₂CH₂CH₂CH₂CH₂CH₂CH₂CN |
| A-714 | CHClCH₂CH₂CH₂CH₂CH₂CH₂CH₂CH₂CH₂CN |
| A-715 | CCl₂CH₂CH₂CH₂CH₂CH₂CH₂CH₂CH₂CH₂CN |
| A-716 | CF₂CH₂CH₂CH₂CH₂CH₂CH₂CH₂CH₂CH₂CN |

The compounds I are suitable as fungicides. They are distinguished by an outstanding effectiveness against a broad spectrum of phytopathogenic fungi from the classes of the Ascomycetes, Deuteromycetes, Oomycetes and Basidiomycetes, especially from the class of the Oomycetes. Some are systemically effective and they can be used in plant protection as foliar fungicides, fungicides for seed dressing and soil fungicides.

They are particularly important in the control of a multitude of fungi on various cultivated plants, such as wheat, rye, barley, oats, rice, corn, grass, bananas, cotton, soybeans, coffee, sugar cane, vines, fruits, ornamental plants, and vegetables, such as cucumbers, beans, tomatoes, potatoes and cucurbits, and on the seeds of these plants.

They are especially suitable for controlling the following plant diseases:
Alternaria species on vegetables, rapeseed, sugar beet and fruit and rice (for example A. solani or A. alternata on potato and other plants),
Aphanomyces species on sugar beet and vegetables,
Bipolaris and Drechslera species on corn, cereals, rice and lawns (for example D. teres on barley, D. tritci-repentis on wheat),
Blumeria graminis (powdery mildew) on cereals,
Botrytis cinerea (gray mold) on strawberries, vegetables, flowers and grapevines,
Bremia lactucae on lettuce,
Cercospora species on corn, soybeans, rice and sugar beet (for example C. beticula on sugar beet),
Cochliobolus species on corn, cereals, rice (for example Cochliobolus sativus on cereals, Cochliobolus miyabeanus on rice),
Colletotricum species on soybeans, cotton and other plants (for example C. acutatum on various plants),
Exserohilum speciea on corn,
Erysiphe cichoracearum and Sphaerotheca fuliginea on cucurbits,
Fusarium and Verticillium species (for example V. dahliae) on various plants (for example F. graminearum on wheat),
Gaeumanomyces graminis on cereals,
Gibberella species on cereals and rice (for example Gibberella fujikuroi on rice),
Grainstaining complex on rice,
Helminthosporium species (for example H. graminicola) on corn and rice,
Michrodochium nivale on cereals,
Mycosphaerella species on cereals, bananas and peanuts (M. graminicola on wheat, M. fijiesis on bananas),
Phakopsara pachyrhizi and Phakopsara meibomiae on soybeans,
Phomopsis species on soybeans, sunflowers and grapevines (P. viticola on grapevines, P. helianthii on sunflowers),
Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucurbits (for example P. cubenis on cucumbers),
Puccinia species on cereals, corn and asparagus (P. triticina and P. striformis on wheat, P. asparagi on asparagus),
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rapeseed, sunflowers, sugar beet, vegetables and other plants,
Rhizoctonia-species (for example R. solani) on cotton, rice, potatoes, lawns, corn, rapeseed, potatoes, sugar beet, vegetables and other plants,
Sclerotinia species (for example S. sclerotiorum) on rapeseed, sunflowers and other plants,
Septoria tritici and Stagonospora nodorum on wheat,
Erysiphe (syn. Uncinula necator) on grapevines,
Setospaeria species on corn and lawns,
Sphacelotheca reilinia on corn,
Thievaliopsis species on soybeans and cotton,
Tilletia species on cereals,
Ustilago species on cereals, corn and sugar beet and
Venturia species (scab) on apples and pears (for example V. inaequalis on apples).

They are particularly suitable for controlling harmful fungi from the class of the Oomycetes, such as Peronospora species, Phytophthora species, Plasmopara viticola and Pseudoperonospora species.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg, preferably 5 to 100 g/100 kg of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I may be present in various crystal modifications which may differ in their biological activity. They also form part of the subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers; if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristearylphenyl polyglycol ethers, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or a water-soluble solvent. As an alternative, wetters or other auxiliaries are added. The active compound dissolves upon dilution with water. A formulation having an active compound content of 10% by weight is obtained in this manner.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added into 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants and wetters as well as silica gel. Dilution with water gives a stable dispersion or solution of the active compound. The active compound content of the formulation is 75% by weight.

### H Gel Formulations

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dustable Powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

### K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity.

The following list of fungicides, in conjunction with which the compounds according to the invention can be used, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethyl-phenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedures described in the following synthesis examples were used to prepare further compounds I by appropriate modification of the starting materials. The compounds thus obtained are listed in the table below, together with physical data.

### Example 1

### Preparation of 4-cyanoundecan-3-one

At −70° C., a solution of 0.495 mol of butyllithium in hexane was added to a solution of 0.45 mol of decanitrile in 300 ml of tetrahydrofuran (THF), the mixture was then stirred at this temperature for about three hours and 0.45 mol of ethyl propionate was added. The mixture was then stirred at 20-25° C. for another about 16 hours, and 200 ml of water were then added and the mixture was acidified with dilute HCl solution. After phase separation, the organic phase was removed, washed with water and dried, and the solvent was removed. This gave 91 g of the title compound.

### Example 2

### Preparation of 7-amino-2-chloro-5-ethyl-6-octyl-[1,2,4]triazolo[1,5-a]pyrimidine [I-1]

A solution of 1.6 g (13.5 mmol) of 5-chloro-1H-[1,2,4]triazol-3-ylamine, 2.8 g (13.5 mmol) of 4-cyanoundecan-3-one from Example 1 and a catalytic amount of p-toluenesulfonic acid in 50 ml of mesitylene was stirred at 180° C. for 4 hours, during which time the water of reaction destilled off. After removal of the solvent by distillation, the residue was digested in a dichloromethane/water mixture at 20 to 25° C. The insoluble components were filtered off and then recrystallized from methanol. This gave 2.9 g of the title compound in the form of colorless crystals of m.p. 202-203° C.

TABLE I
Compounds of the formula I

| No. | R¹ | R² | R³ | A | Phys. Data (m.p. [° C.]) |
|---|---|---|---|---|---|
| I-1 | (CH₂)₇CH₃ | CH₂CH₃ | Cl | N | 202-203 |
| I-2 | (CH₂)₇CH₃ | CH₂CH₃ | NH₂ | N | 216-217 |
| I-3 | (CH₂)₇CH₃ | CH₂CH₃ | C₆H₅ | N | 234-235 |
| I-4 | (CH₂)₇CH₃ | CH₂CH₃ | SCH₃ | N | 233-234 |
| I-5 | (CH₂)₇CH₃ | CH₂CH₃ | CF₃ | N | 176-177 |
| I-6 | (CH₂)₇CH₃ | CH₂CH₃ | SH | N | 275 |
| I-7 | (CH₂)₇CH₃ | CH₂CH₃ | NHCH₃ | N | >280 |
| I-8 | (CH₂)₇CH₃ | CH₂CH₃ | N(CH₃)₂ | N | >280 |
| I-9 | (CH₂)₇CH₃ | CH₂CH₃ | OCH₃ | N | 209-210 |
| I-10 | (CH₂)₇CH₃ | CH₂CH₃ | SO₂CH₃ | N | 163-164 |
| I-11 | (CH₂)₇CH₃ | CH₂CH₃ | S(O)CH₃ | N | 177-178 |
| I-12 | (CH₂)₅CH₃ | CH₂CH₃ | SCH₃ | N | 227-228 |
| I-13 | (CH₂)₆CH₃ | CH₂CH₂CH₃ | SCH₃ | N | 200-201 |
| I-14 | (CH₂)₇CH₃ | CHClCH₃ | NH₂ | N | 189-190 |
| I-15 | (CH₂)₃O(CH₂)₅CH₃ | CH₂CH₃ | NH₂ | N | 183-184 |
| I-16 | (CH₂)₃O(CH₂)₇CH₃ | CH₂OCH₃ | NH₂ | N | 234-237 |
| I-17 | (CH₂)₇CH₃ | CH₂CH₃ | SCH₂C₆H₅ | N | 170-171 |
| I-18 | (CH₂)₇CH₃ | CH₂OCH₃ | NH₂ | N | 260 |
| I-19 | (CH₂)₇CH₃ | CH₂CH₃ | C₆H₅ | CH | 139-140 |
| I-20 | CH₂CH(CH₂CH₃)₂ | CH₂CH₃ | SCH₃ | N | 225-226 |
| I-21 | (CH₂)₇CH₃ | CH₂CH₃ | C₆H₅ | N | 234-235 |
| I-22 | CH₂C≡CCH₃ | CH₂CH₂CH₃ | NH₂ | N | 308-310 |
| I-23 | (CH₂)₃CH═CH₂ | CH₂CH₃ | Cl | N | 185-186 |
| I-24 | CH₂C≡CCH₃ | CH₂CH₂CH₃ | Cl | N | 221-225 |
| I-25 | (CH₂)₄CH₃ | CH₂CH₃ | SCH₃ | N | 227-228 |
| I-26 | (CH₂)₂C₆H₅ | CH₂CH₂-c- | NH₂ | N | 203-205 |
| | | C₆H₁₁ | | | |
| I-27 | (CH₂)₂C₆H₅ | CH₂CH₂-c- | SCH₃ | N | 166-167 |
| | | C₆H₁₁ | | | |
| I-28 | (CH₂)₂C₆H₅ | CH₂CH₂CH(CH₃)₂ | NH₂ | N | 206-207 |
| I-29 | (CH₂)₂C₆H₅ | CH₂CH₂CH(CH₃)₂ | SCH₃ | N | 189-190 |

c-C₆H₁₁ = Cyclohexyl

### Examples of the Action Against Harmful Fungi

The fungicidal action of the compounds of the formula I was demonstrated by the following experiments:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent:emulsifier of 99:1. The mixture was then made up to 100 ml with water. This stock solution was diluted with the solvent/emulsifier/water mixture described to the concentration of active compounds stated below.

### Use Example 1

### Activity Against Late Blight on Tomatoes Caused by Phytophthora infestans, Protective Treatment

Leaves of potted tomato plants were sprayed to runoff point with an aqueous suspension having the concentration of active compound stated below. The next day, the leaves were infected with an aqueous sporangia suspension of Phytophthora infestans. The plants were then placed in a water vapor-saturated chamber at temperatures between 18 and 20° C. After 6 days, the late blight on the untreated, but infected control plants had developed to such an extent that the infection could be determined visually in %.

In this test, the plants which had been treated with 500 ppm of the compound I-2 showed no infection, whereas the untreated plants were 90% infected.

In a further experiment, the plants which had been treated with 250 ppm of the compound I-15 or 1-16 showed an infection of at most 20%, whereas the untreated plants were 100% infected.

### Use Example 2

### Activity Against Peronospora of Grapevines Caused by Plasmopara viticola

Leaves of potted vine were sprayed to runoff point with an aqueous suspension having the concentration of active compound stated below. The next day, the undersides of the leaves were inoculated with an aqueous sporangia suspension of Plasmopara viticola. The vines were then initially placed in a water vapor-saturated chamber at 24° C. for 48 hours and then in a greenhouse at temperatures between 20 and 30° C. for 5 days. After this time, the plants were once more placed in a humid chamber for 16 hours to accelerate the eruption of sporangiophores. The extent of the development of the infection on the undersides of the leaves was then determined visually.

In this test, the plants which had been treated with 500 ppm of the compound I-2, I-7 or I-8 showed an infection of at most 20%, whereas the untreated plants were 90% infected.

In a further experiment, the plants which had been treated with 250 ppm of the compounds I-15, I-16 or I-18 and the plants which had been treated with 63 ppm of the compound I-23 showed an infection of at most 20%, whereas the untreated plants were 100% infected.

1-11. (canceled)

12: A compound of formula I having the following structure: 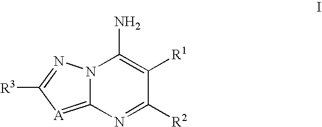
wherein,
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₂-alkenyl or C₁-C₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₂-alkynyl, C₃-C₈-cycloalkyl, C₁-C₁₂-alkoxy, C₁-C₁₂-alkoxy-C₁-C₁₂-alkyl or C₁-C₁₂-alkylthio-C₁-C₁₂-alkyl;
wherein the carbon chains in R¹ and R² can be substituted by one to four identical or different groups R^{a}:
R^{a} is halogen, cyano, hydroxyl, mercapto, C₁-C₁₀-alkyl, C₁-C₁₀-halo-alkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, phenyl, C₁-C₆-alkylphenyl or NR^{A}R^{B}, wherein R^{A} and R^{B} are selected from the group consisting of hydrogen and C₁-C₆-alkyl;
wherein the cyclic groups in R^{a} may be substituted by one to four groups R^{b}:
R^{b} is halogen, cyano, hydroxyl, mercapto, C₁-C₁₀-alkyl, C₁-C₁₀-halo-alkyl, C₂-C₁₀-alkenyl, C₂-C₁₀-alkynyl or C₁-C₆-alkoxy;
R³ is halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2; and
A is N or CR^{x}, wherein R^{x} is hydrogen, halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2.

13: The compound of claim 12, wherein:
R¹ is C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl, wherein the carbon chains may be substituted by one to four identical or different groups R^{a}, and
R² is methyl, ethyl, n-propyl, n-butyl, n-pentyl, n-hexyl, n-hepyl, n-octyl, n-nonyl, n-decyl, methoxymethyl or ethoxymethyl.

14: The compound of claim 12, wherein:
R³ is halogen, cyano, NH₂, hydroxyl, mercapto, C₂-C₆-alkyl, C₁-C₆-haloalkyl, C₃-C₈-cycloalkyl, C₁-C₆-alkoxy or C₁-C₆-alkylthio.

15: The compound of claim 13, wherein:
R³ is halogen, cyano, NH₂, hydroxyl, mercapto, C₂-C₆-alkyl, C₁-C₆-haloalkyl, C₃-C₈-cycloalkyl, C₁-C₆-alkoxy or C₁-C₆-alkylthio.

16: The compound of claim 12, wherein A is N.

17: The compound of claim 13, wherein A is N.

18: The compound of claim 14, wherein A is N.

19: The compound of claim 15, wherein A is N.

20: A method of preparing compounds of the formula I having the following structure: 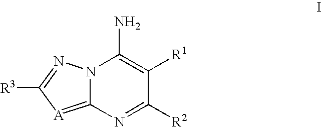
wherein,
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl or C₁-C₁₂-alkylthio-C₁-C₁₂-alkyl;
wherein the carbon chains in R¹ and R² may be substituted by one to four identical or different groups R^{a}:
R^{a} is halogen, cyano, hydroxyl, mercapto, C₁-C₁₀-alkyl, C₁-C₁₀-halo-alkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, phenyl, C₁-C₆-alkylphenyl or NR^{A}R^{B}, wherein R^{A} and R^{B} are selected from the group consisting of hydrogen and C₁-C₆-alkyl;
wherein the cyclic groups in R^{a} may be substituted by one to four groups R^{b}:
R^{b} is halogen, cyano, hydroxyl, mercapto, C₁-Clo-alkyl, C₁-C₁₀-halo-alkyl, C₂-C₁₀-alkenyl, C₂-C₁₀-alkynyl or C₁-C₆-alkoxy;
R³ is halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2; and
A is N or CR^{x}, wherein R^{x} is hydrogen, halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxy-carbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2,
said method comprising,
a) contacting a compound of formula II, 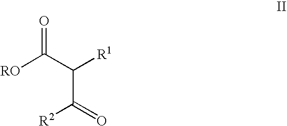
in which R is C₁-C₄-alkyl and R¹ and R² are as described above, with a compound of formula III 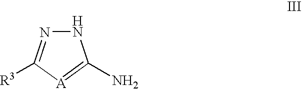
wherein R³ is as described above, to yield a compound of formula IV 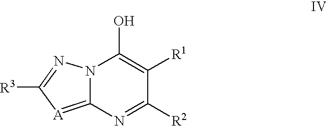
wherein R¹, R² and R³ are as described above;
b) halogenating said compound of formula IV to yield a compound of formula V, 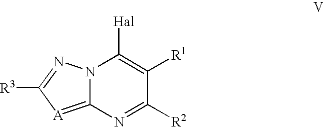
in which Hal is chlorine or bromine and R¹, R² and R³ are as described above; and
c) contacting a compound of formula V with ammonia,
wherein a compound of formula I is prepared.

21: A method of preparing a compound of formula I having the following structure: 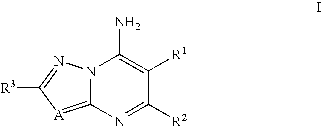
wherein,
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₂-alkyl, C₁-C₁₂-alkoxy-C₂-C₂-alkenyl or C₁-C₁₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl or C₁-C₁₂-alkylthio-C₁-C₁₂-alkyl;
wherein the carbon chains in R¹ and R² may be substituted by one to four identical or different groups R^{a}:
R^{a} is halogen, cyano, hydroxyl, mercapto, C₁-C₁₀-alkyl, C₁-C₁₀-halo-alkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, phenyl, C₁-C₆-alkylphenyl or NR^{A}R^{B}, wherein R^{A} and R^{B} are selected from the group consisting of hydrogen and C₁-C₆-alkyl;
wherein the cyclic groups in R^{a} may be substituted by one to four groups R^{b}:
R^{b} is halogen, cyano, hydroxyl, mercapto, C₁-C₁₀-alkyl, C₁-C₁₀-halo-alkyl, C₂-C₁₀-alkenyl, C₂-C₁₀-alkynyl or C₁-C₆-alkoxy;
R³ is halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2; and
A is N or CR^{x}, wherein R^{x} is hydrogen, halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxy-carbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2,
said method comprising, contacting a compound of formula VI 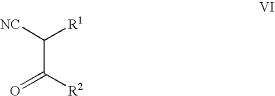
wherein, R¹ and R² are as described above, with a compound of formula III 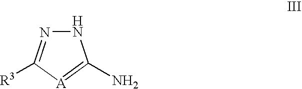
wherein, R³ is as described above,
wherein a compound of formula I is prepared.

22: A fungicidal composition comprising a solid or liquid carrier and a compound of the formula I having the following structure: 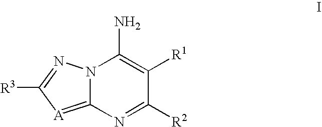
wherein,
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₂-alkyl, C₁-C₁₂-alkoxy-C₂-C₂-alkenyl or C₁-C₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl or C₁-C₁₂-alkylthio-C₁-C₁₂-alkyl;
wherein the carbon chains in R¹ and R² may be substituted by one to four identical or different groups R^{a}:
R^{a} is halogen, cyano, hydroxyl, mercapto, C₁-C₁₀-alkyl, C₁-C₁₀-halo-alkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, phenyl, C₁-C₆-alkylphenyl or NR^{A}R^{B}, wherein R^{A}, R^{B} are selected from the group consisting of hydrogen and C₁-C₆-alkyl;
wherein the cyclic groups in R^{a} may be substituted by one to four groups R^{b}:
R^{b} is halogen, cyano, hydroxyl, mercapto, C₁-C₁₀-alkyl, C₁-C₁₀-halo-alkyl, C₂-C₁₀-alkenyl, C₂-C₁₀-alkynyl or C₁-C₆-alkoxy;
R³ is halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2; and
A is N or CR^{x}, wherein R^{x} is hydrogen, halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxy-carbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2.

23: The composition of claim 22, comprising the compound of formula I wherein,
R¹ is C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl, wherein the carbon chains may be substituted by one to four identical or different groups R^{a}, and
R² is methyl, ethyl, n-propyl, n-butyl, n-pentyl, n-hexyl, n-hepyl, n-octyl, n-nonyl, n-decyl, methoxymethyl or ethoxymethyl.

24: The composition of claim 22, comprising the compound of formula I wherein,
R³ is halogen, cyano, NH₂, hydroxyl, mercapto, C₂-C₆-alkyl, C₁-C₆-haloalkyl, C₃-C₈-cycloalkyl, C₁-C₆-alkoxy or C₁-C₆-alkylthio.

25: The composition of claim 22, comprising the compound of formula I wherein, A is N.

26: The composition of claim 22, further comprising another active compound.

27: A seed comprising the compound of the formula I having the following structure: 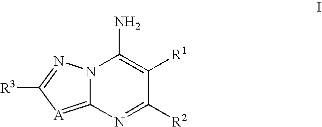
wherein,
R¹ is hydrogen, halogen, cyano, C₁-C₁₄-alkyl, C₁-C₁₄-haloalkyl, C₂-C₁₂-alkenyl, C₂-C₂-alkynyl, C₃-C₈-cycloalkyl, C₁-C₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl or C₁-C₁₂-alkylthio-C₁-C₁₂-alkyl;
wherein the carbon chains in R¹ and R² may be substituted by one to four identical or different groups R^{a}:
R^{a} is halogen, cyano, hydroxyl, mercapto, C₁-C₁₀-alkyl, C₁-C₁₀-halo-alkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, phenyl, C₁-C₆-alkylphenyl or NR^{A}R^{B}, wherein R^{A}, R^{B} are selected from the group consisting of hydrogen and C₁-C₆-alkyl;
wherein the cyclic groups in R^{a} may be substituted by one to four groups R^{b}:
R^{b} is halogen, cyano, hydroxyl, mercapto, C₁-C₁₀-alkyl, C₁-C₁₀-halo-alkyl, C₂-C₁₀-alkenyl, C₂-C₁₀-alkynyl or C₁-C₆-alkoxy;
R³ is halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2; and
A is N or CR^{x}, wherein R^{x} is hydrogen, halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2,
in amounts of 1 to 1000 g per 100 kg of seed.

28: A method for controlling phytopathogenic harmful fungi comprising, contacting the fungi or the materials, plants, the soil or seed to be protected against fungal attack with an effective amount of the compound of the formula I having the following structure: 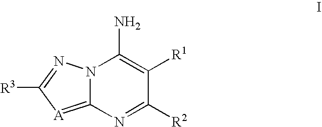
wherein,
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl or C₁-C₁₂-alkylthio-C₁-C₁₂-alkyl;
wherein the carbon chains in R¹ and R² may be substituted by one to four identical or different groups R^{a}:
R^{a} is halogen, cyano, hydroxyl, mercapto, C₁-C₁₀-alkyl, C₁-C₁₀-halo-alkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, phenyl, C₁-C₆-alkylphenyl or NR^{A}R^{B}, wherein R^{A}, R^{B} are selected from the group consisting of hydrogen and C₁-C₆-alkyl;
wherein the cyclic groups in R^{a} may be substituted by one to four groups R^{b}:
R^{b} is halogen, cyano, hydroxyl, mercapto, C₁-C₁₀-alkyl, C₁-C₁₀-halo-alkyl, C₂-C₁₀-alkenyl, C₂-C₁₀-alkynyl or C₁-C₆-alkoxy;
R³ is halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2; and
A is N or CR^{x}, wherein R^{x} is hydrogen, halogen, cyano, NR^{A}R^{B}, wherein R^{A} and R^{B} are as describe above, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—, wherein m is 0, 1 or 2.

29: The method of claim 28, wherein said compound of formula I comprises a compound wherein,
R¹ is C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl, wherein the carbon chains may be substituted by one to four identical or different groups R^{a}, and
R² is methyl, ethyl, n-propyl, n-butyl, n-pentyl, n-hexyl, n-hepyl, n-octyl, n-nonyl, n-decyl, methoxymethyl or ethoxymethyl.

30: The method of claim 28, wherein said compound of formula I comprises a compound wherein,
R³ is halogen, cyano, NH₂, hydroxyl, mercapto, C₂-C₆-alkyl, C₁-C₆-haloalkyl, C₃-C₈-cycloalkyl, C₁-C₆-alkoxy or C₁-C₆-alkylthio.

31: The method of claim 28, wherein said compound of formula I comprises a compound wherein, A is N.
